(19) United States
(12) Patent Application Publication
(10) Pub. No.: US 2020/0361930 A1
(43) Pub. Date: Nov. 19, 2020

(54) BIFUNCTIONAL MOLECULES THAT DEGRADE EGFR

(71) Applicants: Hoffmann-La Roche Inc., Little Falls, NJ (US); C4 Therapeutics, Inc., Watertown, MA (US)

(72) Inventors: Martin Duplessis, Somerville, MA (US); Georg Jaeschke, Basel (CH); Bernd Kuhn, Reinach BL (CH); Kiel Lazarski, Boston, MA (US); Yanke Liang, Belmont, MA (US); Yvonne Alice Nagel, Basel (CH); Antonio Ricci, Biel-Benken (CH); Daniel Rueher, Raedersdorf (FR); Sandra Steiner, Sursee (CH)

(73) Assignees: Hoffmann-La Roche Inc., Little Falls, NJ (US); C4 Therapeutics, Inc., Watertown, MA (US)

(21) Appl. No.: 16/984,987

(22) Filed: Aug. 4, 2020

Related U.S. Application Data
(63) Continuation of application No. PCT/EP2019/052585, filed on Feb. 4, 2019.

(30) Foreign Application Priority Data
Feb. 5, 2018 (EP) 18155128.4

(51) Int. Cl. C07D 417/14; C07D 401/14

(52) U.S. Cl. CPC C07D 417/14; C07D 401/14

(57) ABSTRACT

The present invention provides compounds that cause specifically the degradation of EGFR via the targeted ubiquitination of EGFR protein and subsequent proteasomal degradation. The present compounds are useful for the treatment of various cancers.

### CROSS-REFERENCE TO RELATED APPLICATIONS

This application is a continuation of International Application No. PCT/EP2019/052585, filed in the International Patent Cooperation Treaty, European Receiving Office on Feb. 4, 2019, which claims the benefit of European Patent Application No. 18155128.4, filed Feb. 5, 2018. The entirety of these applications are hereby incorporated by reference herein for all purposes.

### FIELD OF THE INVENTION

Present invention provides compounds that cause specifically the degradation of EGFR via the targeted ubiquitination of EGFR protein and subsequent proteasomal degradation. The present compounds are useful for the treatment of various cancers.

### BACKGROUND OF THE INVENTION

The field of targeted protein degradation promoted by small molecules has been intensively studied over the last years. See Collins et al., Biochem J, 2017, 474(7), 1127-47.

Protein degradation plays a role in various cellular functions, i.e. the concentrations of regulatory proteins are adjusted through degradation into small peptides to maintain health and productivity of the cells.

Cereblon is a protein that forms an E3 ubiquitin ligase complex, which ubiquinates various other proteins. Cereblon is known as primary target for anticancer thalidomide analogs. A higher expression of cereblon has been linked to the efficiency of thalidomide analogs in cancer therapy.

In the recent years, a few bifunctional compounds have been described as useful modulators of targeted ubiquitination, e.g. WO2013020557, WO2013063560, WO 2013106643, WO2015160845, WO2016011906, WO2016105518, WO2017007612, WO2017024318 and W2017117473.

EGFR inhibitors, in particular selective inhibitors of T790M containing EGFR mutants have been described for instance in WO2014081718, WO2014210354 and Zhou et al., “Novel mutant-selective EGFR kinase inhibitors against EGFR T790M”, NATURE, (20091224), vol. 462, no. 7276, doi:10.1038/nature08622, ISSN 0028-0836, pages 1070-1074.

Bifunctional molecules for degradation of EGFR are described for instance in WO2017185036.

However, there is still an ongoing need for effective treatment of cancers.

### SUMMARY OF THE INVENTION

Present invention provides compounds that cause specifically the degradation of EGFR via the targeted ubiquitination of EGFR protein and subsequent proteasomal degradation. Present compounds are useful for the treatment of various cancers. Present compounds bind to the ubiquitously expressed E3 ligase protein cereblon (CRBN) on one hand and alter the substrate specificity of the CRBN E3 ubiquitin ligase complex, resulting in the recruitment and ubiquitination of EGFR. The present compounds are also selective inhibitors of T790M containing EGFR mutants.

Present invention provides compounds of formula I, or a pharmaceutically acceptable salt thereof,

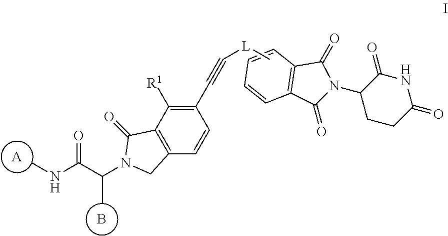

wherein the substituents and variables are as described below and in the claims, or a pharmaceutically acceptable salt thereof.

The present compounds are useful for the therapeutic and/or prophylactic treatment of cancer.

The compounds of present invention can further be used as part of bifunctional compounds that comprise the compounds of present invention as E3 Ubiquitin Ligase moiety that is linked to a moiety that binds to a target protein where the target protein is proximate to the ubiquitin ligase to effect degradation of said protein.

### DETAILED DESCRIPTION OF THE INVENTION

The present invention provides a compound of formula I and their pharmaceutically acceptable salts thereof, the preparation of the above mentioned compounds, medicaments containing them and their manufacture as well as the use of the above mentioned compounds in the therapeutic and/or prophylactic treatment of cancer.

The following definitions of the general terms used in the present description apply irrespectively of whether the terms in question appear alone or in combination with other groups.

Unless otherwise stated, the following terms used in this application, including the specification and claims, have the definitions given below. It must be noted that, as used in the specification and the appended claims, the singular forms “a”, “an,” and “the” include plural referents unless the context clearly dictates otherwise.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-propyl), 1,2-dimethyl-propyl and the like. A specific group is methyl.

The term “halogen”, alone or in combination with other groups, denotes chloro (Cl), iodo (I), fluoro (F) and bromo (Br). A specific group is F.

The term “hydroxy” means —OH.

The term “heterocyclyl” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Specific “heterocyclyl” are saturated monocyclic rings systems of 4-6 ring atoms, comprising 1-2 ring heteroatoms that are N. Examples for monocyclic saturated heterocycloalkyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl, or dihydropyranyl. Specific groups are piperazinyl and piperidinyl.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Particular “heteroaryl” have 6 rings atoms, comprising one N. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl. A specific group is pyridinyl.

A “piperazinyl” being part of the subunit “L” is connected at both ends via the respective “N”.

A “piperidinyl” being part of the subunit “L” is connected at one ends via the “N”.

The subunit “L” is linked via a “C” to the alkynyl moiety of the molecule and with a “N” to the isoindolinyl moiety of the molecule, for example, when L is aryl-(CH₂)₁₋₂-heterocyclyl-C(═O)—(CH₂)₁₋₁₀—NH—, then the compound of formula I is

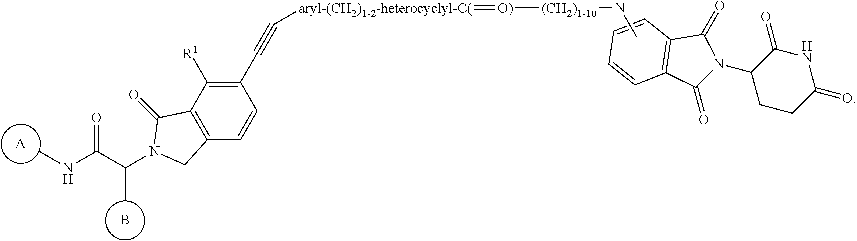

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The term “a pharmaceutically acceptable salt” refers to a salt that is suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid. A specific acid is trifluoroacetic acid.

The terms “pharmaceutically acceptable auxiliary substance” refer to carriers and auxiliary substances such as diluents or excipients that are compatible with the other ingredients of the formulation.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “pharmaceutically acceptable excipient” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

Whenever a chiral carbon is present in a chemical structure, it is intended that all stereoisomers associated with that chiral carbon are encompassed by the structure as pure stereoisomers as well as mixtures thereof.

The invention also provides pharmaceutical compositions, methods of using, and methods of preparing the aforementioned compounds.

All separate embodiments may be combined.

E1: One embodiment of the invention relates to a compound of formula I, or a pharmaceutically acceptable salt thereof,

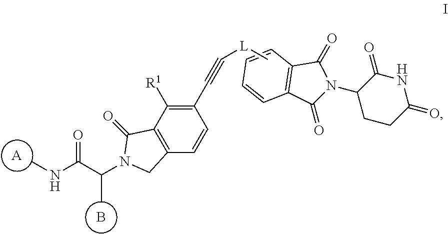

wherein
L is selected from the group consisting of
   i) -aryl-(CH₂)₁₋₂-heterocyclyl-C(═O)—(CH₂)₁₋₁₀—NH—, in particular
      a. -phenyl-(CH₂)₁₋₂-piperazinyl-C(═O)—(CH₂)₁₋₁₀—NH—;
   ii) -heteroaryl-C(═O)—NH-heterocyclyl-C(═O)—(CH₂)₁₋₁₀—NH—, in particular
      a. -pyridinyl-C(═O)—NH-piperidyl-C(═O)—(CH₂)₁₋₁₀—NH—;
   iii) -heteroaryl-(CH₂)₁₋₂-heterocyclyl-C(═O)—(CH₂)₁₋₁₀—NH—, in particular
      a. -pyridinyl-(CH₂)₁₋₂-piperazinyl-C(═O)—(CH₂)₁₋₁₀—NH—;
   iv) -heteroaryl-C(═O)—NH-heterocyclyl-(CH₂)₁₋₁₀—NH—, in particular
      a. -pyridinyl-C(═O)—NH-piperidyl-(CH₂)₁₋₁₀—NH—;
   v) -heteroaryl-C(═O)—NH-heterocyclyl-(CH₂)₁₋₁₀-heterocyclyl-, in particular
      a. -pyridinyl-C(═O)—NH-piperidyl-(CH₂)₁₋₁₀-piperidyl-; and
   vi) -heteroaryl-(CH₂)₁₋₂-heterocyclyl-C(═O)—(CH₂)₁₋₁₀-heterocyclyl-, in particular
      a. -pyridinyl-(CH₂)₁₋₂-piperidyl-C(═O)—(CH₂)₁₋₁₀-piperidyl-, or
      b. -pyridinyl-(CH₂)₁₋₂-piperazinyl-C(═O)—(CH₂)₁₋₁₀-piperidyl-.
         whereby each aryl or heteroaryl moiety can be independently substituted by
a. halogen, in particular F, or
b. C₁₋₆alkyl, in particular methyl;
R¹ is H;
A is heteroaryl, in particular
   a. thiazolyl, or
   b. pyridinyl;
B is aryl, in particular phenyl, which aryl is
   a. unsubstituted, or
   b. substituted by 1-2 substituents individually selected from
      i. halogen, in particular F,
      ii. C₁₋₆alkyl, in particular methyl, and
      iii. hydroxy.

E2: The compound of formula I, or pharmaceutically acceptable salts thereof, as described herein, wherein L is selected from the group consisting of
i) -5F-pyridinyl-C(═O)—NH-piperidyl-(CH₂)₄—NH—,
ii) -phenyl-(CH₂)1-piperazinyl-C(═O)—(CH₂)₃—NH—,
iii) -phenyl-(CH₂)1-piperazinyl-C(═O)—(CH₂)₅—NH—,
iv) -pyridinyl-(CH₂)1-piperazinyl-C(═O)—(CH₂)₁-piperidyl-,
v) -pyridinyl-(CH₂)1-piperazinyl-C(═O)—(CH₂)₃—NH—,
vi) -pyridinyl-C(═O)—NH-piperidyl-(CH₂)₁-piperidyl-,
vii) -pyridinyl-C(═O)—NH-piperidyl-(CH₂)₄—NH—,
viii) -pyridinyl-C(═O)—NH-piperidyl-C(═O)—(CH₂)₁—NH—, and
ix) -pyridinyl-C(═O)—NH-piperidyl-C(═O)—(CH₂)₃—NH—.

E3: The compound of formula I, or pharmaceutically acceptable salts thereof, as described herein, wherein A is thiazolyl.

E4: The compound of formula I, or pharmaceutically acceptable salts thereof, as described herein, wherein B is phenyl.

E5: The compound of formula I, or pharmaceutically acceptable salts thereof, as described herein, selected from the group consisting of
(2RS)-2-[6-[2-[4-[[4-[4-[[2-[(3RS)-2,6-Dioxo-3-piperidyl]-1,3-dioxo-isoindolin-4-yl]amino]butanoyl]piperazin-1-yl]methyl]phenyl]ethynyl]-1-oxo-isoindolin-2-yl]-2-phenyl-N-thiazol-2-yl-acetamide,
(2RS)-2-[6-[2-[4-[[4-[6-[[2-[(3RS)-2,6-Dioxo-3-piperidyl]-1,3-dioxo-isoindolin-4-yl]amino]hexanoyl]piperazin-1-yl]methyl]phenyl]ethynyl]-1-oxo-isoindolin-2-yl]-2-phenyl-N-thiazol-2-yl-acetamide,
(2RS)-2-[6-[2-[6-[[4-[2-[1-[2-[(3RS)-2,6-Dioxo-3-piperidyl]-1,3-dioxo-isoindolin-4-yl]-4-piperidyl]acetyl]piperazin-1-yl]methyl]-3-pyridyl]ethynyl]-1-oxo-isoindolin-2-yl]-2-phenyl-N-(2-pyridyl)acetamide,
(2RS)-2-[6-[2-[6-[[4-[4-[[2-[(3RS)-2,6-Dioxo-3-piperidyl]-1,3-dioxo-isoindolin-4-yl]amino]butanoyl]piperazin-1-yl]methyl]-3-pyridyl]ethynyl]-1-oxo-isoindolin-2-yl]-2-phenyl-N-thiazol-2-yl-acetamide,
N-[1-[[1-[2-[(3RS)-2,6-Dioxo-3-piperidyl]-1,3-dioxo-isoindolin-5-yl]-4-piperidyl]methyl]-4-piperidyl]-5-[2-[3-oxo-2-[(1RS)-2-oxo-1-phenyl-2-(2-pyridylamino)ethyl]isoindolin-5-yl]ethynyl]pyridine-2-carboxamide,
N-[1-[2-[[2-[(3RS)-2,6-Dioxo-3-piperidyl]-1,3-dioxo-isoindolin-4-yl]amino]acetyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide,
N-[1-[4-[[2-[(3RS)-2,6-Dioxo-3-piperidyl]-1,3-dioxo-isoindolin-4-yl]amino]butanoyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide,
N-[1-[4-[[2-[(3RS)-2,6-Dioxo-3-piperidyl]-1,3-dioxo-isoindolin-4-yl]amino]butyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide,
N-[1-[4-[[2-[(3RS)-2,6-Dioxo-3-piperidyl]-1,3-dioxo-isoindolin-4-yl]amino]butanoyl]-4-piperidyl]-5-[2-[2-[(1RS)-1-(5-fluoro-2-hydroxy-phenyl)-2-oxo-2-(thiazol-2-ylamino)ethyl]-3-oxo-isoindolin-5-yl]ethynyl]pyridine-2-carboxamide,
N-[1-[4-[[2-[(3RS)-2,6-Dioxo-3-piperidyl]-1,3-dioxo-isoindolin-4-yl]amino]butyl]-4-piperidyl]-3-fluoro-5-[2-[3-oxo-2-[(1RS)-2-oxo-1-phenyl-2-(thiazol-2-ylamino)ethyl]isoindolin-5-yl]ethynyl]pyridine-2-carboxamide,
N-[1-[4-[[2-[(3RS)-2,6-Dioxo-3-piperidyl]-1,3-dioxo-isoindolin-4-yl]amino]butyl]-4-piperidyl]-5-[2-[3-oxo-2-[(1RS)-2-oxo-1-phenyl-2-(2-pyridylamino)ethyl]isoindolin-5-yl]ethynyl]pyridine-2-carboxamide, and
N-[1-[4-[[2-[(3RS)-2,6-Dioxo-3-piperidyl]-1,3-dioxo-isoindolin-4-yl]amino]butyl]-4-piperidyl]-5-[2-[2-[(1RS)-1-(5-fluoro-2-hydroxy-phenyl)-2-oxo-2-(thiazol-2-ylamino)ethyl]-3-oxo-isoindolin-5-yl]ethynyl]pyridine-2-carboxamide.

1. The compound according to any one of claims 1-5 for use as a medicament.

E6: The compound as described herein, or pharmaceutically acceptable salts thereof, for the therapeutic and/or prophylactic treatment of cancer.

E7: Use of the compound as described herein, or pharmaceutically acceptable salts thereof, for the therapeutic and/or prophylactic treatment of cancer.

E8: A pharmaceutical composition comprising a compound as described herein, and a therapeutically inert carrier.

E9: A certain embodiment of the invention refers to the compound of formula I, or pharmaceutically acceptable salts thereof, as described herein, for use as medicament.

E10: A certain embodiment of the invention relates to the compound of formula I as described herein, or a pharmaceutically acceptable salt thereof, for use as therapeutically active substance.

E11: A certain embodiment of the invention relates to the compound of formula I as described herein, or a pharmaceutically acceptable salt thereof, for the use in the therapeutic and/or prophylactic treatment of cancer.

E12: A certain embodiment of the invention relates to the compound of formula I as described herein, or a pharmaceutically acceptable salt thereof, for the manufacture of a medicament for the therapeutic and/or prophylactic treatment of cancer.

E13: A certain embodiment of the invention relates to a pharmaceutical composition comprising the compound of formula I as described herein, or a pharmaceutically acceptable salt thereof, and a pharmaceutically acceptable auxiliary substance.

E14: A certain embodiment of the invention relates to a method for the therapeutic and/or prophylactic treatment of cancer, by administering the compound of formula I as described herein, or a pharmaceutically acceptable salt thereof, to a patient.

Furthermore, the invention includes all optical isomers, i.e. diastereoisomers, diastereomeric mixtures, racemic mixtures, all their corresponding enantiomers and/or tautomers as well as their solvates of the compounds of formula I.

The compounds of formula I may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains>90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

The compounds of formula I may be prepared in accordance with the schemes described in the examples. The starting material is commercially available or may be prepared in accordance with known methods.

The preparation of compounds of formula I is further described in more detail in the scheme below.

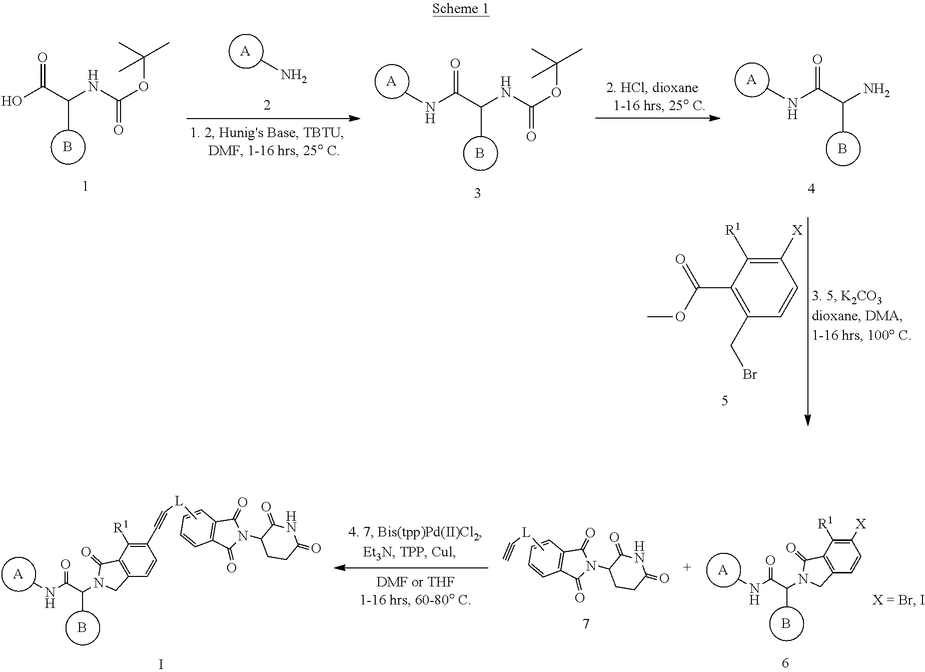

An isoindoline-acetylene based compound of general formula I can be obtained for example by amide coupling with an appropriately substituted acid of formula 1 and an appropriately substituted amine of formula 2 with a coupling agent such as TBTU to yield the desired amide derivatives of formula 3. Deprotection followed by ring cyclization with a iodo or bromo substituted methyl 2-(bromomethyl)benzoate of formula 5 yields the desired isoindoline 6. Sonogashira coupling with an appropriate substituted acetlyne of formula 7 forms the desired isoindoline-acetylene based compound of general formula I (scheme 1).

Generally speaking, the sequence of steps used to synthesize the compounds of formula I can also be modified in certain cases.

Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC. Racemic mixtures of chiral synthetic intermediates may also be separated using chiral HPLC.

Salts of Compounds of Formula I

In cases where the compounds of formula I are basic they may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

Insofar as their preparation is not described in the examples, the compounds of formula I as well as all intermediate products can be prepared according to analogous methods or according to the methods set forth herein. Starting materials are commercially available, known in the art or can be prepared by methods known in the art or in analogy thereto.

It will be appreciated that the compounds of general formula I in this invention may be derivatised at functional groups to provide derivatives which are capable of conversion back to the parent compound in vivo.

### Pharmacological Tests

The compounds of formula I and their pharmaceutically acceptable salts possess valuable pharmacological properties. The compounds were investigated in accordance with the test given hereinafter.

EGFR Degradation Assay (Cellular)

### Generation of BaF3 EGFR Mutant Cell Lines

The BaF3 parental line was purchased from DSMZ and grown in RPMI media supplemented with 10% FBS and 10 ng/mL interleukin 3 (IL-3) (Thermo Fisher Scientific). EGFR mutants (T790M/L853R, T790M/L853R/C797S) were cloned into the pCDH lentiviral vector (SystemBio) under the control of a PGK promoter and confirmed by DNA sequencing. The resulting gene expression vector for each mutant was mixed with packaging vectors and cotransfected into 2×10E6 HEK293T cells (ATCC) in 10 mL of DMEM media to generate lentiviral particles according to the manufacturers protocol (Origene).

Three days post-transfection, the viral supernatant was harvested and filtered. In one well of a 12-well plate, 0.5 mL of viral supernatant was added to 2E6 Ba/F3 cells contained in 1.5 mL of RPMI media including 10% FBS, 10 ng/mL IL-3, and 5 μg/mL polybrene (Invitrogen). The plate was centrifuged at 2,000 rpm for 1 hour at room temperature and infected cells were kept in a tissue culture incubator overnight at 37° C. The cells were washed once in fresh BaF3 media and reseeded at 0.5E6 cells/well of a 12-well plate in media supplemented with 0.5 μg/mL puromycin. The cells were maintained in this media for 3 weeks. IL-3-independent, EGFR mutant transformed cells were routinely maintained in RPMI medium supplemented with 10% FBS.

### Materials

RPMI 1640 no-phenol red medium and fetal bovine serum (FBS) were purchased from Gibco (Grand Island, N.Y., USA). EGFR total kit and EGFR phospho-Y1068 kit were purchased from Cisbio (Bedford, Mass., USA). BaF3 EGFR mutant cell lines (EGFR T790M/L858R/C797S) cell line was generated in house, according to the protocol reported above. Cell culture flasks and 384-well microplates were acquired from VWR (Radnor, Pa., USA).

### EGFR Degradation Analysis

EGFR degradation was determined based on quantification of FRET signal using EGFR total kit. The FRET signal detected correlates with total EGFR protein level in cells. Briefly, test compounds were added to the 384-well plate from a top concentration of 1 μM with 11 points, half log titration in quadruplicates. Then, BaF3 EGFR mutant cell lines (EGFR T790M/L858R/C797S) were added into 384-well plates at a cell density of 10000 cells per well. The plates were kept at 37° C. with 5% CO₂ for 4 hours. After 4-hour incubation, 4× lysis buffer was added to the cells, and then then microplate was agitated on plate shaker at 500 rpm for 30 minutes at room temperature. Next, total EGFR antibody solution was added to the cells and the cells were incubated for another 4 hours at room temperature. Finally, FRET signal was acquired on EnVision™ Multilabel Reader (PerkinElmer, Santa Clara, Calif., USA). The cells treated in the absence of the test compound were the negative control and lysis buffer with antibody solution only were the positive control.

TABLE 1
IC₅₀ value (BaF3 EGFR T790M/L858R/C797S degradation)

| | | | IC₅₀ |
|---|---|---|---|
| Ex | Structure | Name | [nM] |
| 1 | 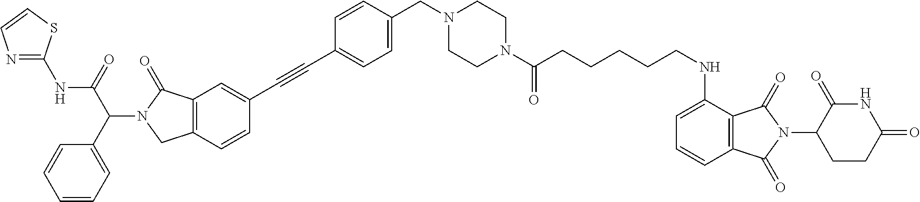 [SMILES: O=C1CCC(N2C(=O)c3cccc(NCCCCCC(=O)N4CCN(Cc5ccc(C#Cc6ccc7c(c6)C(=O)N(C(C(=O)Nc6nccs6)c6ccccc6)C7)cc5)CC4)c3C2=O)C(=O)N1] | (2RS)-2-[6-[2-[4-[[4-[6- [[2-[(3RS)-2,6-Dioxo-3- piperidyl]-1,3-dioxo- isoindolin-4- yl]amino]hexanoyl] piperazin-1- yl]methyl]phenyl]ethynyl]- 1-oxo-isoindolin-2- yl]-2-phenyl-N-thiazol- 2-yl-acetamide | 40 |
| 2 | 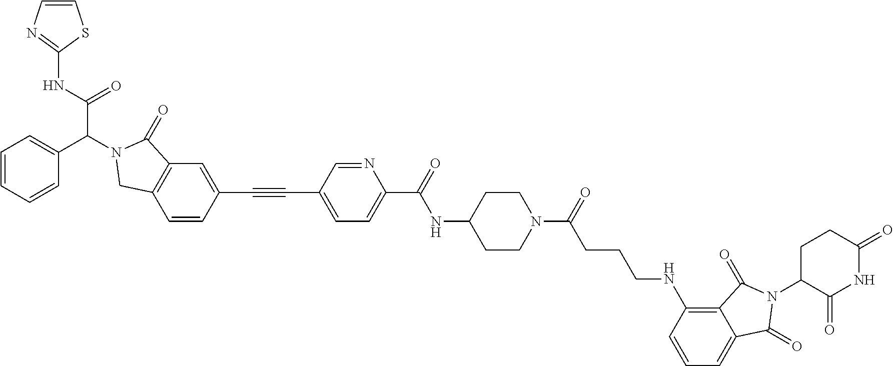 [SMILES: O=C1CCC(N2C(=O)c3cccc(NCCCC(=O)N4CCC(NC(=O)c5ccc(C#Cc6ccc7c(c6)C(=O)N(C(C(=O)Nc6nccs6)c6ccccc6)C7)cn5)CC4)c3C2=O)C(=O)N1] | N-[1-[4-[[2-[(3RS)-2,6- Dioxo-3-piperidyl]-1,3- dioxo-isoindolin-4- yl]amino]butanoyl]-4- piperidyl]-5-[2-[3-oxo- 2-[(1RS)-2-oxo-1- phenyl-2-(thiazol-2- ylamino)ethyl]isoindolin- 5-yl]ethynyl]pyridine- 2-carboxamide | 21 |
| 3 | 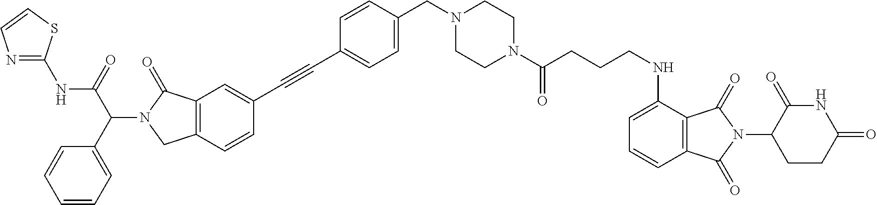 [SMILES: O=C1CCC(N2C(=O)c3cccc(NCCCC(=O)N4CCN(Cc5ccc(C#Cc6ccc7c(c6)C(=O)N(C(C(=O)Nc6nccs6)c6ccccc6)C7)cc5)CC4)c3C2=O)C(=O)N1] | (2RS)-2-[6-[2-[4-[[4-[4- [2-[(3RS)-2,6-Dioxo-3- piperidyl]-1,3-dioxo- isoindolin-4- yl]amino]butanoyl] piperazin-1- yl]methyl]phenyl]ethynyl]- 1-oxo-isoindolin-2- yl]-2-phenyl-N-thiazol- 2-yl-acetamide | 35 |
| 4 | 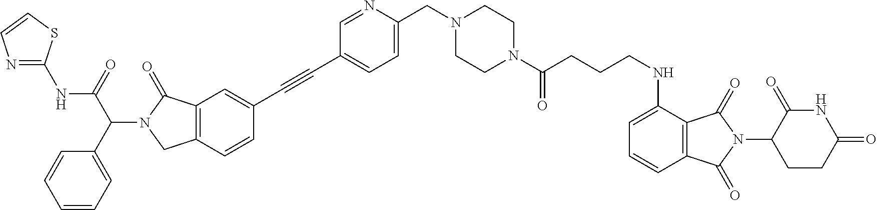 [SMILES: O=C1CCC(N2C(=O)c3cccc(NCCCC(=O)N4CCN(Cc5ccc(C#Cc6ccc7c(c6)C(=O)N(C(C(=O)Nc6nccs6)c6ccccc6)C7)cn5)CC4)c3C2=O)C(=O)N1] | (2RS)-2-[6-[2-[6-[[4-[4- [[2-[(3RS)-2,6-Dioxo-3- piperidyl]-1,3-dioxo- isoindolin-4- yl]amino]butanoyl] piperazin-1-yl]methyl]-3- pyridyl]ethynyl]-1-oxo- isoindolin-2-yl]-2- phenyl-N-thiazol-2-yl- acetamide | 14 |
| 5 | 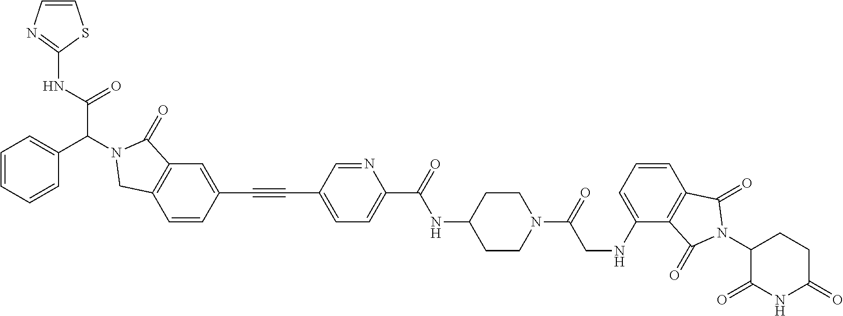 [SMILES: O=C1CCC(N2C(=O)c3cccc(NCC(=O)N4CCC(NC(=O)c5ccc(C#Cc6ccc7c(c6)C(=O)N(C(C(=O)Nc6nccs6)c6ccccc6)C7)cn5)CC4)c3C2=O)C(=O)N1] | N-[1-[2-[[2-[(3RS)-2,6- Dioxo-3-piperidyl]-1,3- dioxo-isoindolin-4- yl]amino]acetyl]-4- piperidyl]-5-[2-[3-oxo- 2-[(1RS)-2-oxo-1- phenyl-2-(thiazol-2- ylamino)ethyl]isoindolin- 5-yl]ethynyl]pyridine- 2-carboxamide | 18 |
| 6 | 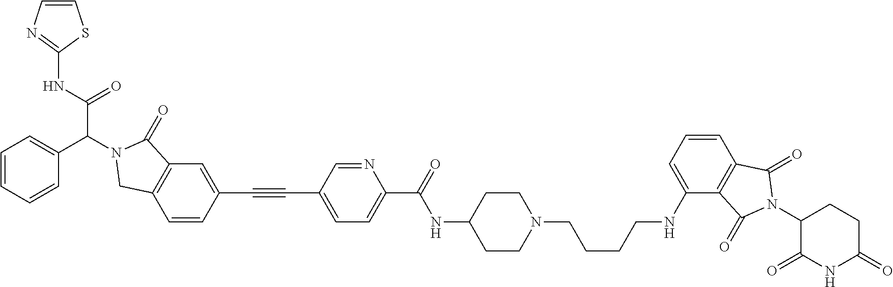 [SMILES: O=C1CCC(N2C(=O)c3cccc(NCCCCN4CCC(NC(=O)c5ccc(C#Cc6ccc7c(c6)C(=O)N(C(C(=O)Nc6nccs6)c6ccccc6)C7)cn5)CC4)c3C2=O)C(=O)N1] | N-[1-[4-[[2-[(3RS)-2,6- Dioxo-3-piperidyl]-1,3- dioxo-isoindolin-4- yl]amino]butyl]-4- piperidyl]-5-[2-[3-oxo- 2-[(1RS)-2-oxo-1- phenyl-2-(thiazol-2- ylamino)ethyl]isoindolin- 5-yl]ethynyl]pyridine- 2-carboxamide | 21 |
| 7 | 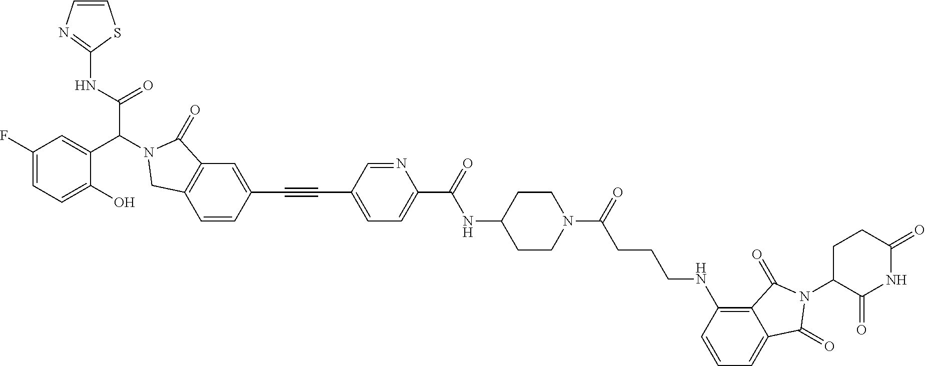 [SMILES: O=C1CCC(N2C(=O)c3cccc(NCCCC(=O)N4CCC(NC(=O)c5ccc(C#Cc6ccc7c(c6)C(=O)N(C(C(=O)Nc6nccs6)c6cc(F)ccc6O)C7)cn5)CC4)c3C2=O)C(=O)N1] | N-[1-[4-[[2-[(3RS)-2,6- Dioxo-3-piperidyl]-1,3- dioxo-isoindolin-4- yl]amino]butanoyl]-4- piperidyl]-5-[2-[2- [(1RS)-1-(5-fluoro-2- hydroxy-phenyl)-2-oxo- 2-(thiazol-2- ylamino)ethyl]-3-oxo- isoindolin-5- yl]ethynyl]pyridine-2- carboxamide | 12 |
| 8 | 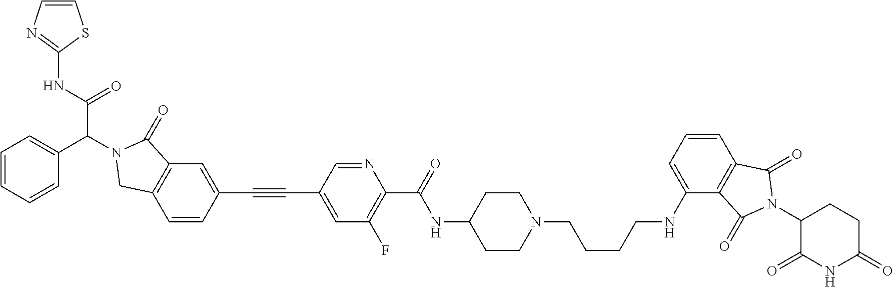 [SMILES: O=C1CCC(N2C(=O)c3cccc(NCCCCN4CCC(NC(=O)c5ncc(C#Cc6ccc7c(c6)C(=O)N(C(C(=O)Nc6nccs6)c6ccccc6)C7)cc5F)CC4)c3C2=O)C(=O)N1] | N-[1-[4-[[2-[(3RS)-2,6- Dioxo-3-piperidyl]-1,3- dioxo-isoindolin-4- yl]amino]butyl]-4- piperidyl]-3-fluoro-5-[2- [3-oxo-2-[(1RS)-2-oxo- 1-phenyl-2-(thiazol-2- ylamino)ethyl]isoindolin- 5-yl]ethynyl]pyridine- 2-carboxamide | 20 |
| 9 | 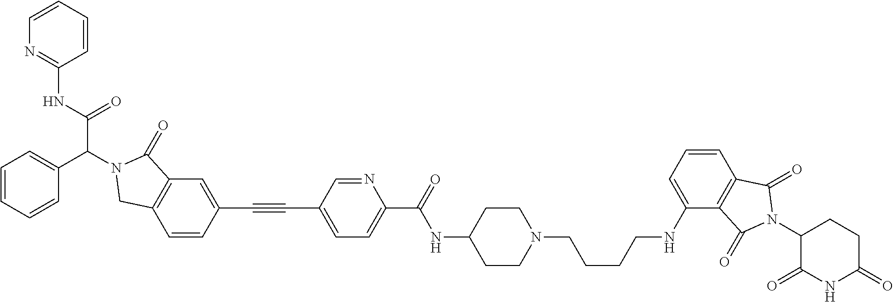 [SMILES: O=C1CCC(N2C(=O)c3cccc(NCCCCN4CCC(NC(=O)c5ccc(C#Cc6ccc7c(c6)C(=O)N(C(C(=O)Nc6ccccn6)c6ccccc6)C7)cn5)CC4)c3C2=O)C(=O)N1] | N-[1-[4-[[2-[(3RS)-2,6- Dioxo-3-piperidyl]-1,3- dioxo-isoindolin-4- yl)amino]butyl]-4- piperidyl]-5-[2-[3-oxo- 2-[(1RS)-2-oxo-1- phenyl-2-(2- pyridylamino)ethyl] isoindolin-5- yl]ethynyl]pyridine-2- carboxamide | 52 |
| 10 | 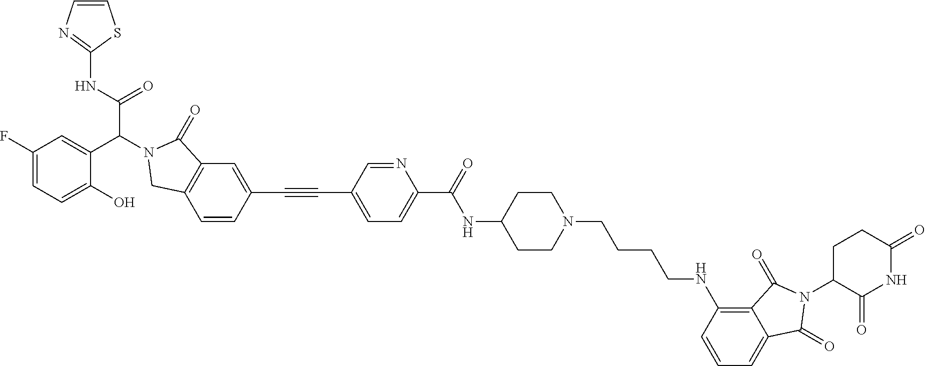 [SMILES: O=C1CCC(N2C(=O)c3cccc(NCCCCN4CCC(NC(=O)c5ccc(C#Cc6ccc7c(c6)C(=O)N(C(C(=O)Nc6nccs6)c6cc(F)ccc6O)C7)cn5)CC4)c3C2=O)C(=O)N1] | N-[1-[4-[[2-[(3RS)-2,6- Dioxo-3-piperidyl]-1,3- dioxo-isoindolin-4- yl]amino]butyl]-4- piperidyl]-5-[2-[2- [(1RS)-1-(5-fluoro-2- hydroxy-phenyl)-2-oxo- 2-(thiazol-2- ylamino)ethyl]-3-oxo- isoindolin-5- yl]ethynyl]pyridine-2- carboxamide | 13 |
| 11 | 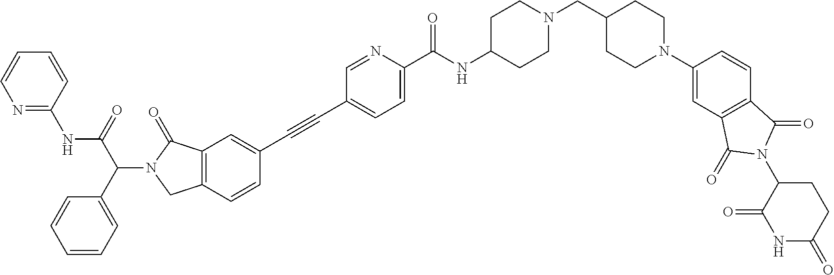 [SMILES: O=C1CCC(N2C(=O)c3ccc(N4CCC(CN5CCC(NC(=O)c6ccc(C#Cc7ccc8c(c7)C(=O)N(C(C(=O)Nc7ccccn7)c7ccccc7)C8)cn6)CC5)CC4)cc3C2=O)C(=O)N1] | N-[1-[[1-[2-[(3RS)-2,6- Dioxo-3-piperidyl]-1,3- dioxo-isoindolin-5-yl]-4- piperidyl]methyl]-4- piperidyl]-5-[2-[3-oxo- 2-[(1RS)-2-oxo-1- phenyl-2-(2- pyridylamino)ethyl] isoindolin-5- yl]ethynyl]pyridine-2- carboxamide | 48 |
| 12 | 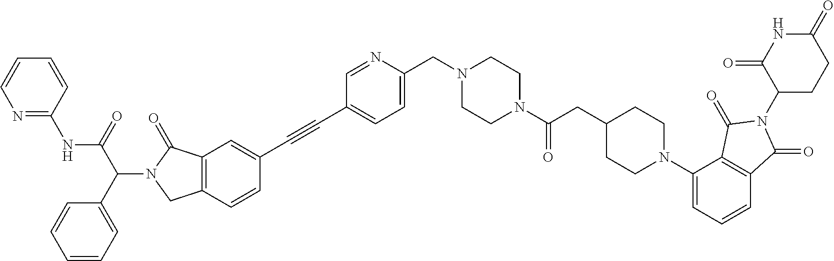 [SMILES: O=C1CCC(N2C(=O)c3cccc(N4CCC(CC(=O)N5CCN(Cc6ccc(C#Cc7ccc8c(c7)C(=O)N(C(C(=O)Nc7ccccn7)c7ccccc7)C8)cn6)CC5)CC4)c3C2=O)C(=O)N1] | (2RS)-2-[6-[2-[6-[[4-[2- [1-[2-[(3RS)-2,6-Dioxo- 3-piperidyl]-1,3-dioxo- isoindolin-4-yl]-4- piperidyl]acetyl]piperazin- 1-yl]methyl]-3- pyridyl]ethynyl]-1-oxo- isoindolin-2-yl]-2- phenyl-N-(2- pyridyl)acetamide | 38 |

### Pharmaceutical Compositions

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragees, hard and soft gelatin capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also provided by the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula I. Examples of compositions according to the invention are:

### Example A

### Tablets of the Following Composition are Manufactured in the Usual Manner:

TABLE 2
possible tablet composition

| | mg/tablet | | | |
|---|---|---|---|---|
| ingredient | 5 | 25 | 100 | 500 |
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Drythe granulesat 50° C.
3. Pass the granules through suitable milling equipment.
4. Add ingredient 5 and mix for three minutes; compress on a suitable press.

### Example B-1

### Capsules of the Following Composition are Manufactured:

TABLE 3
possible capsule ingredient composition

| | mg/capsule | | | |
|---|---|---|---|---|
| ingredient | 5 | 25 | 100 | 500 |
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

### Soft Gelatin Capsules of the Following Composition are Manufactured:

TABLE 4
possible soft gelatin capsule ingredient composition

| ingredient | mg/capsule |
|---|---|
| Compound of formula I | 5 |
| Yellow wax | 8 |
| Hydrogenated Soya bean oil | 8 |
| Partially hydrogenated plant oils | 34 |
| Soya bean oil | 110 |
| Total | 165 |

TABLE 5
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

### Suppositories of the Following Composition are Manufactured:

TABLE 6
possible suppository composition

| ingredient | mg/supp. |
|---|---|
| Compound of formula I | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

### Injection Solutions of the Following Composition are Manufactured:

TABLE 7
possible injection solution composition

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

### Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

### Sachets of the Following Composition are Manufactured:

TABLE 8
possible sachet composition

| ingredient | mg/sachet |
|---|---|
| Compound of formula I | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula I is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Experimental Part

The following examples are provided for illustration of the invention. They should not be considered as limiting the scope of the invention, but merely as being representative thereof.

### Example 1

### (2RS)-2-[6-[2-[4-[[4-[6-[[2-[(3RS)-2,6-Dioxo-3-piperidyl]-1,3-dioxo-isoindolin-4-yl]amino]hexanoyl]piperazin-1-yl]methyl]phenyl]ethynyl]-1-oxo-isoindolin-2-yl]-2-phenyl-N-thiazol-2-yl-acetamide

### Step 1: tert-Butyl N-[(1RS)-2-oxo-1-phenyl-2-(thiazol-2-ylamino)ethyl]carbamate

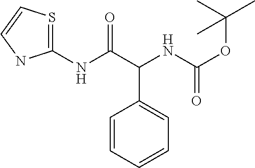

(2RS)-2-(tert-Butoxycarbonylamino)-2-phenyl-acetic acid (9.5 g, 37.8 mmol) was dissolved in 75 ml of ethyl acetate and 10 ml of DMF. Thiazol-2-amine (3.79 g, 37.8 mmol, 1 equiv.), Hunig's base (14.7 g, 19.8 ml, 113 mmol, 3 equiv.) and Propylphosphonic anhydride solution (50% in ethyl acetate) (36.1 g, 33.8 ml, 56.7 mmol, 1.5 equiv.) were added drop wise at room temperature. The mixture was stirred at room temperature for 30 minutes. The reaction mixture was extracted with saturated NaHCO₃-solution and two times with ethyl acetate. The organic layers were extracted with water, dried over sodium sulfate and evaporated to dryness. The desired tert-butyl N-[(1RS)-2-oxo-1-phenyl-2-(thiazol-2-ylamino)ethyl]carbamate (12 g, 95% yield) was obtained as a light yellow solid, MS: m/e=334.5 (M+H⁺).

### Step 2: (2RS)-2-Amino-2-phenyl-N-thiazol-2-yl-acetamide hydrochloride

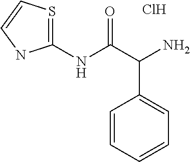

tert-Butyl N-[(1RS)-2-oxo-1-phenyl-2-(thiazol-2-ylamino)ethyl]carbamate (Example 1, step 1) (12 g, 37 mmol) was dissolved in 100 ml of MeOH and HCl (4N in dioxane) (27.7 ml, 111 mmol, 3 equiv.) was added at room temperature. The mixture was stirred for 5 hours at room temperature. The reaction mixture was evaporated to dryness and used directly in the next step. The desired (2RS)-2-amino-2-phenyl-N-thiazol-2-yl-acetamide hydrochloride (quantitative yield) was obtained as a grey solid, MS: m/e=234.4 (M+H⁺).

### Step 3: (2RS)-2-(6-Iodo-1-oxo-isoindolin-2-yl)-2-phenyl-N-thiazol-2-yl-acetamide

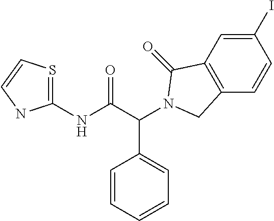

(2RS)-2-Amino-2-phenyl-N-thiazol-2-yl-acetamide hydrochloride (Example 1, step 2) (1.22 g, 4.51 mmol) was dissolved in 15 ml of dioxane and 2.5 ml of DMA. Methyl 2-(bromomethyl)-5-iodobenzoate (CAS 1310377-56-0) (1.6 g, 4.51 mmol, 1 equiv.) and triethylamine (2.28 g, 3.14 ml, 22.5 mmol, 5 equiv.) were added at room temperature. The mixture was stirred at 100° C. for 2 hours. The reaction mixture was extracted with water and two times with ethyl acetate. The organic layers were extracted with brine, dried over sodium sulfate and evaporated to dryness. The crude product was purified by flash chromatography on a silica gel column eluting with an ethyl acetate:heptane 0:100 to 100:0 gradient to obtain the desired (2RS)-2-(6-iodo-1-oxo-isoindolin-2-yl)-2-phenyl-N-thiazol-2-yl-acetamide (870 mg, 41% yield) as a yellow solid, MS: m/e=475.9 (M+H⁺).

### Step 4: tert-Butyl 4-[(4-ethynylphenyl)methyl]piperazine-1-carboxylate

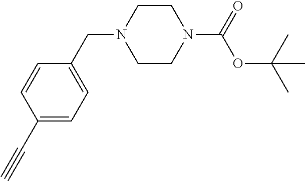 [SMILES: C#Cc1ccc(CN2CCN(C(=O)OC(C)(C)C)CC2)cc1]

4-Ethynylbenzaldehyde (400 mg, 3.07 mmol) was dissolved in 15 ml of dichloromethane and tert-butyl piperazine-1-carboxylate (690 mg, 3.69 mmol, 1.2 equiv.) followed by sodium triacetoxyhydroborate (780 mg, 3.69 mmol, 1.2 equiv.) were added at room temperature. The mixture was stirred at room temperature for 16 hours. The reaction mixture was extracted with water and two times with dichloromethane. The organic layers were extracted with brine, dried over sodium sulfate and evaporated to dryness. The crude product was purified by flash chromatography on a silica gel column eluting with an ethyl acetate:heptane 0:100 to 50:50 gradient to obtain the desired tert-butyl 4-(4-ethynylbenzyl)piperazine-1-carboxylate (670 mg, 73% yield) as a colorless oil, MS: m/e=301.5 (M+H⁺).

### Step 5: tert-Butyl 4-[[4-[2-[3-oxo-2-[(1RS)-2-oxo-1-phenyl-2-(thiazol-2-ylamino)ethyl]isoindolin-5-yl]ethynyl]phenyl]methyl]piperazine-1-carboxylate

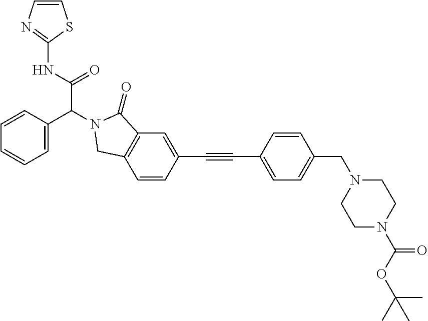 [SMILES: CC(C)(C)OC(=O)N1CCN(Cc2ccc(C#Cc3ccc4c(c3)C(=O)N(C(C(=O)Nc3nccs3)c3ccccc3)C4)cc2)CC1]

(2RS)-2-(6-Iodo-1-oxo-isoindolin-2-yl)-2-phenyl-N-thiazol-2-yl-acetamide (Example 1, step 3) (600 mg, 1.26 mmol) and tert-butyl 4-(4-ethynylbenzyl)piperazine-1-carboxylate (Example 1, step 4) (664 mg, 2.21 mmol, 1.75 equiv.) were dissolved in 12 ml of THF. Triethylamine (383 mg, 0.53 ml, 3.79 mmol, 3 equiv.), bis-(triphenylphosphine)-palladium(II)dichloride (87 mg, 0.126 mmol, 0.1 equiv.), triphenylphosphine (66 mg, 0.25 mmol, 0.2 equiv.) and copper(I)iodide (24 mg, 0.126 mmol, 0.1 equiv.) were added and the mixture was stirred for 16 hours at 60° C. The reaction mixture was extracted with water and two times with ethyl acetate. The organic layers were extracted with brine, dried over sodium sulfate and evaporated to dryness. The crude product was purified by flash chromatography on a silica gel column eluting with a dichloromethane:methanol 100:0 to 90:10 gradient. The desired tert-butyl 4-[[4-[2-[3-oxo-2-[(1RS)-2-oxo-1-phenyl-2-(thiazol-2-ylamino)ethyl]isoindolin-5-yl]ethynyl]phenyl]methyl]piperazine-1-carboxylate (quantitative yield) was obtained as an orange solid, MS: m/e=646.6 (M+H⁺).

### Step 6: (2RS)-2-[1-Oxo-6-[2-[4-(piperazin-1-ylmethyl)phenyl]ethynyl]isoindolin-2-yl]-2-phenyl-N-thiazol-2-yl-acetamide hydrochloride

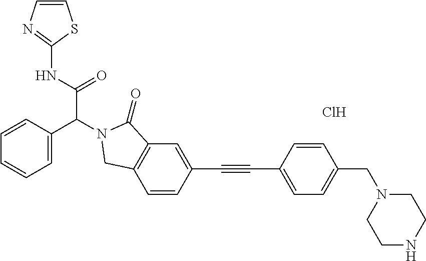 [SMILES: O=C(Nc1nccs1)C(c1ccccc1)N1Cc2ccc(C#Cc3ccc(CN4CCNCC4)cc3)cc2C1=O ; Cl]

The title compound was obtained as a light brown solid, MS: m/e=546.5 (M+H⁺), using chemistry similar to that described in Example 1, step 2 starting from tert-butyl 4-[[4-[2-[3-oxo-2-[(1RS)-2-oxo-1-phenyl-2-(thiazol-2-ylamino)ethyl]isoindolin-5-yl]ethynyl]phenyl]methyl]piperazine-1-carboxylate (Example 1, step 5).

### Step 7: 6-[[2-[(3RS)-2,6-Dioxo-3-piperidyl]-1,3-dioxo-isoindolin-4-yl]amino]hexanoic acid

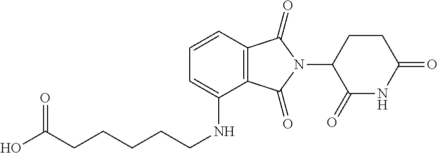 [SMILES: O=C(O)CCCCCNc1cccc2c1C(=O)N(C1CCC(=O)NC1=O)C2=O]

A mixture of 6-aminohexanoic acid (1.7 g, 13.03 mmol, 1.2 equiv.), 2-[(3RS)-2,6-dioxo-3-piperidyl]-4-fluoro-isoindoline-1,3-dione (CAS 835616-60-9) (3 g, 10.86 mmol), Hunig's base (5.7 ml, 32.58 mmol, 3 equiv.) in 50 ml of DMSO was stirred at 100° C. for 16 hours. Water (500 ml) was added to the reaction mixture and extracted four times with ethyl acetate (200.0 ml each). The combined organic layers were washed with brine, dried over sodium sulfate and concentrated to give a residue. The crude product was purified by flash chromatography on a silica gel column eluting with a petroleum ether:ethyl acetate 3:1 to 0:1 gradient and trituration in dichloromethane to obtain the desired 6-[[2-[(3RS)-2,6-dioxo-3-piperidyl]-1,3-dioxo-isoindolin-4-yl]amino]hexanoic acid (1.4 g, 31% yield) as a green solid, MS: m/e=388.1 (M+H⁺).

### Step 8: (2RS)-2-[6-[2-[4-[[4-[6-[[2-[(3RS)-2,6-Dioxo-3-piperidyl]-1,3-dioxo-isoindolin-4-yl]amino]hexanoyl]piperazin-1-yl]methyl]phenyl]ethynyl]-1-oxo-isoindolin-2-yl]-2-phenyl-N-thiazol-2-yl-acetamide

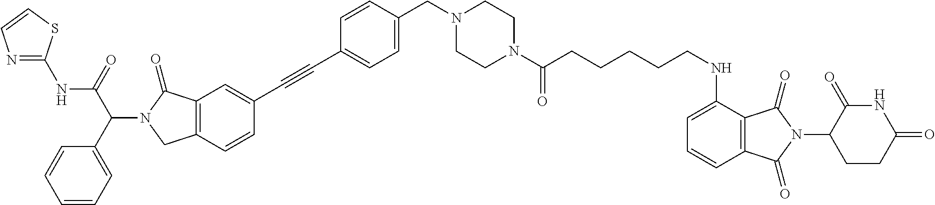 [SMILES: O=C1CCC(N2C(=O)c3cccc(NCCCCCC(=O)N4CCN(Cc5ccc(C#Cc6ccc7c(c6)C(=O)N(C(C(=O)Nc6nccs6)c6ccccc6)C7)cc5)CC4)c3C2=O)C(=O)N1]

The title compound was obtained as a yellow solid, MS: m/e=917.9 (M+H⁺), using chemistry similar to that described in Example 1, step 1 starting from (2RS)-2-[1-oxo-6-[2-[4-(piperazin-1-ylmethyl)phenyl]ethynyl]isoindolin-2-yl]-2-phenyl-N-thiazol-2-yl-acetamide hydrochloride (Example 1, step 6) and 6-[[2-[(3RS)-2,6-dioxo-3-piperidyl]-1,3-dioxo-isoindolin-4-yl]amino]hexanoic acid (Example 1, step 7).

### Example 2

### N-[1-[4-[[2-[(3RS)-2,6-Dioxo-3-piperidyl]-1,3-dioxo-isoindolin-4-yl]amino]butanoyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide

### Step 1: Methyl 5-[2-[3-oxo-2-[(1S)-2-oxo-1-phenyl-2-(thiazol-2-ylamino)ethyl]isoindolin-5-yl]ethynyl]pyridine-2-carboxylate

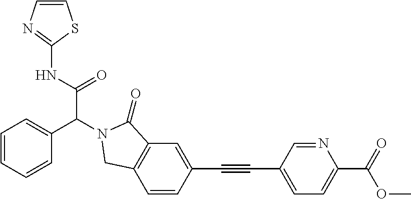 [SMILES: COC(=O)c1ccc(C#Cc2ccc3c(c2)C(=O)N(C(C(=O)Nc2nccs2)c2ccccc2)C3)cn1]

The title compound was obtained as a white solid, MS: m/e=509.4 (M+H⁺), using chemistry similar to that described in Example 1, step 5 starting from (2RS)-2-(6-iodo-1-oxo-isoindolin-2-yl)-2-phenyl-N-thiazol-2-yl-acetamide (Example 1, step 3) and methyl 5-ethynylpicolinate.

### Step 2: 5-[2-[3-Oxo-2-[(1RS)-2-oxo-1-phenyl-2-(thiazol-2-ylamino)ethyl]isoindolin-5-yl]ethynyl]pyridine-2-carboxylic acid

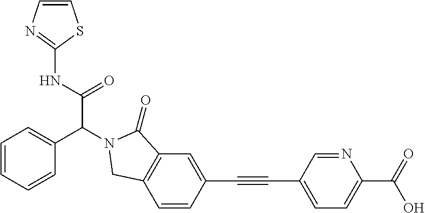 [SMILES: O=C(O)c1ccc(C#Cc2ccc3c(c2)C(=O)N(C(C(=O)Nc2nccs2)c2ccccc2)C3)cn1]

Methyl 5-[2-[3-oxo-2-[(1S)-2-oxo-1-phenyl-2-(thiazol-2-ylamino)ethyl]isoindolin-5-yl]ethynyl]pyridine-2-carboxylate (Example 2, step 1) (900 mg, 1.77 mmol) was dissolved in 9 ml of THF and 3 ml of MeOH and sodium hydroxide (M) (3.54 ml, 3.54 mmol, 2 equiv.) was added. The mixture was stirred for 2 hours at room temperature. 5 ml of 1M KHSO₄ solution were added and the formed precipitate was filtered off, washed with water and dried. The desired 5-[2-[3-oxo-2-[(1RS)-2-oxo-1-phenyl-2-(thiazol-2-ylamino)ethyl]isoindolin-5-yl]ethynyl]pyridine-2-carboxylic acid (862 mg, 99% yield) was obtained as a white solid, MS: m/e=495.3 (M+H⁺).

### Step 3: tert-Butyl 4-[[5-[2-[3-oxo-2-[(1RS)-2-oxo-1-phenyl-2-(thiazol-2-ylamino)ethyl]isoindolin-5-yl]ethynyl]pyridine-2-carbonyl]amino]piperidine-1-carboxylate

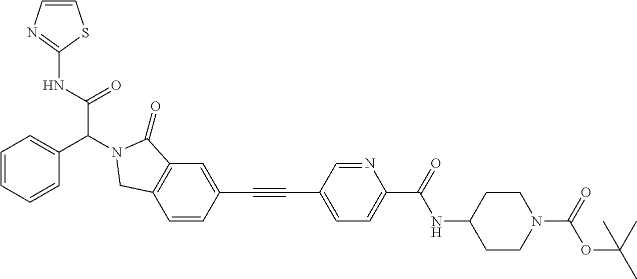 [SMILES: CC(C)(C)OC(=O)N1CCC(NC(=O)c2ccc(C#Cc3ccc4c(c3)C(=O)N(C(C(=O)Nc3nccs3)c3ccccc3)C4)cn2)CC1]

The title compound was obtained as a light yellow foam, MS: m/e=677.4 (M+H⁺), using chemistry similar to that described in Example 1, step 1 starting from 5-[2-[3-oxo-2-[(RS)-2-oxo-1-phenyl-2-(thiazol-2-ylamino)ethyl]isoindolin-5-yl]ethynyl]pyridine-2-carboxylic acid (Example 2, step 2) and tert-butyl 4-aminopiperidine-1-carboxylate.

### Step 4: 5-[2-[3-Oxo-2-[(1RS)-2-oxo-1-phenyl-2-(thiazol-2-ylamino)ethyl]isoindolin-5-yl]ethynyl]-N-(4-piperidyl)pyridine-2-carboxamide hydrochloride

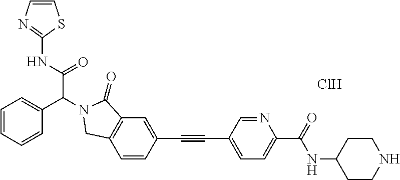 [SMILES: O=C(NC1CCNCC1)c1ccc(C#Cc2ccc3c(c2)C(=O)N(C(C(=O)Nc2nccs2)c2ccccc2)C3)cn1 ; Cl]

The title compound was obtained as a light yellow semi-solid, MS: m/e=577.4 (M+H⁺), using chemistry similar to that described in Example 1, step 2 starting from tert-butyl 4-[[5-[2-[3-oxo-2-[(1RS)-2-oxo-1-phenyl-2-(thiazol-2-ylamino)ethyl]isoindolin-5-yl]ethynyl]pyridine-2-carbonyl]amino]piperidine-1-carboxylate (Example 2, step 3).

### Step 5: 4-[[2-[(3RS)-2,6-Dioxo-3-piperidyl]-1,3-dioxo-isoindolin-4-yl]amino]butanoic acid

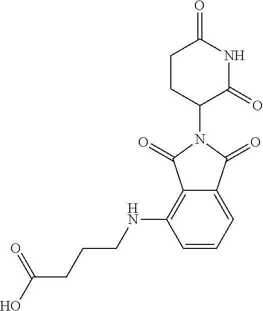 [SMILES: O=C(O)CCCNc1cccc2c1C(=O)N(C1CCC(=O)NC1=O)C2=O]

The title compound was obtained as a light green solid, MS: m/e=360.1 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 4-aminobutyric acid.

### Step 6: N-[1-[4-[[2-[(3RS)-2,6-Dioxo-3-piperidyl]-1,3-dioxo-isoindolin-4-yl]amino]butanoyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide

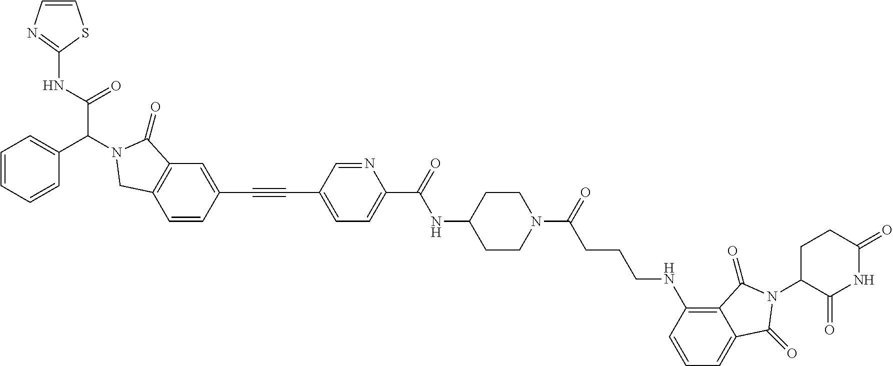 [SMILES: O=C1CCC(N2C(=O)c3cccc(NCCCC(=O)N4CCC(NC(=O)c5ccc(C#Cc6ccc7c(c6)C(=O)N(C(C(=O)Nc6nccs6)c6ccccc6)C7)cn5)CC4)c3C2=O)C(=O)N1]

The title compound was obtained as a yellow solid, MS: m/e=918.5 (M+H⁺), using chemistry similar to that described in Example 1, step 1 starting from 5-[2-[3-oxo-2-[(RS)-2-oxo-1-phenyl-2-(thiazol-2-ylamino)ethyl]isoindolin-5-yl]ethynyl]-N-(4-piperidyl)pyridine-2-carboxamide hydrochloride (Example 2, step 4) and 4-[[2-[(3RS)-2,6-dioxo-3-piperidyl]-1,3-dioxo-isoindolin-4-yl]amino]butanoic acid (Example 2, step 5).

### Example 3

### (2RS)-2-[6-[2-[4-[[4-[4-[[2-[(3RS)-2,6-Dioxo-3-piperidyl]-1,3-dioxo-isoindolin-4-yl]amino]butanoyl]piperazin-1-yl]methyl]phenyl]ethynyl]-1-oxo-isoindolin-2-yl]-2-phenyl-N-thiazol-2-yl-acetamide

The title compound was obtained as a yellow solid, MS: m/e=889.5 (M+H⁺), using chemistry similar to that described in Example 1, step 1 starting from (2RS)-2-[1-oxo-6-[2-[4-(piperazin-1-ylmethyl)phenyl]ethynyl]isoindolin-2-yl]-2-phenyl-N-thiazol-2-yl-acetamide hydrochloride (Example 1, step 6) and 4-[[2-[(3RS)-2,6-dioxo-3-piperidyl]-1,3-dioxo-isoindolin-4-yl]amino]butanoic acid (Example 2, step 5).

### Example 4

### (2RS)-2-[6-[2-[6-[[4-[4-[[2-[(3RS)-2,6-Dioxo-3-piperidyl]-1,3-dioxo-isoindolin-4-yl]amino]butanoyl]piperazin-1-yl]methyl]-3-pyridyl]ethynyl]-1-oxo-isoindolin-2-yl]-2-phenyl-N-thiazol-2-yl-acetamide

### Step 2: tert-Butyl 4-[(5-ethynyl-2-pyridyl)methyl]piperazine-1-carboxylate e

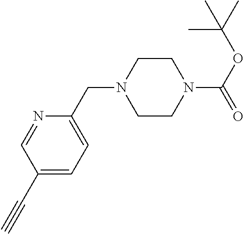 [SMILES: C#Cc1ccc(CN2CCN(C(=O)OC(C)(C)C)CC2)nc1]

The title compound was obtained as a brown oil, MS: m/e=302.2 (M+H⁺), using chemistry similar to that described in Example 1, step 4 starting from 5-ethynylpicolinaldehyde and tert-butyl piperazine-1-carboxylate.

### Step 2: (2RS)-2-[1-Oxo-6-[2-[6-(piperazin-1-ylmethyl)-3-pyridyl]ethynyl]isoindolin-2-yl]-2-phenyl-N-thiazol-2-yl-acetamide hydrochloride

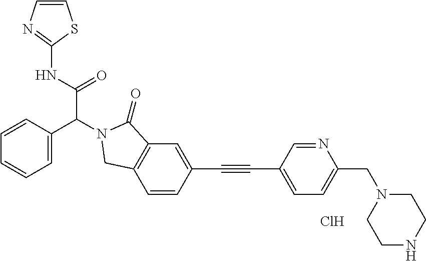 [SMILES: O=C(Nc1nccs1)C(c1ccccc1)N1Cc2ccc(C#Cc3ccc(CN4CCNCC4)nc3)cc2C1=O ; Cl]

The title compound was obtained as an orange solid, MS: m/e=549.4 (M+H⁺), using chemistry similar to that described in Example 1, step 5 and 6 starting from (2RS)-2-(6-iodo-1-oxo-isoindolin-2-yl)-2-phenyl-N-thiazol-2-yl-acetamide (Example 1, step 3) and tert-butyl 4-[(5-ethynyl-2-pyridyl)methyl]piperazine-1-carboxylate (Example 4, step 1).

### Step 2: (2RS)-2-[6-[2-[6-[[4-[4-[[2-[(3RS)-2,6-Dioxo-3-piperidyl]-1,3-dioxo-isoindolin-4-yl]amino]butanoyl]piperazin-1-yl]methyl]-3-pyridyl]ethynyl]-1-oxo-isoindolin-2-yl]-2-phenyl-N-thiazol-2-yl-acetamide

The title compound was obtained as a yellow solid, MS: m/e=888.6 (M−H⁺), using chemistry similar to that described in Example 1, step 1 starting from (2RS)-2-[1-oxo-6-[2-[6-(piperazin-1-ylmethyl)-3-pyridyl]ethynyl]isoindolin-2-yl]-2-phenyl-N-thiazol-2-yl-acetamide hydrochloride (Example 4, step 2) and 4-[[2-[(3RS)-2,6-dioxo-3-piperidyl]-1,3-dioxo-isoindolin-4-yl]amino]butanoic acid (Example 2, step 5).

### Example 5

### N-[1-[2-[[2-[(3RS)-2,6-Dioxo-3-piperidyl]-1,3-dioxo-isoindolin-4-yl]amino]acetyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide

### Step 1: tert-Butyl 2-[[2-[(3RS)-2,6-dioxo-3-piperidyl]-1,3-dioxo-isoindolin-4-yl]amino]acetate

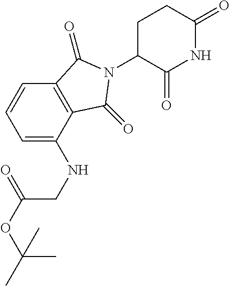 [SMILES: CC(C)(C)OC(=O)CNc1cccc2c1C(=O)N(C1CCC(=O)NC1=O)C2=O]

The title compound was obtained as a yellow solid, MS: m/e=332.1 (M+H⁺-tBu), using chemistry similar to that described in Example 1, step 7 starting from 2-[(3RS)-2,6-dioxo-3-piperidyl]-4-fluoro-isoindoline-1,3-dione (CAS 835616-60-9) and glycine tert-butyl ester hydrochloride.

### Step 1: 2-[[2-[(3RS)-2,6-Dioxo-3-piperidyl]-1,3-dioxo-isoindolin-4-yl]amino]acetic acid

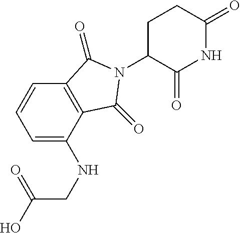 [SMILES: O=C(O)CNc1cccc2c1C(=O)N(C1CCC(=O)NC1=O)C2=O]

The title compound was obtained as a yellow solid, MS: m/e=332.1 (M+H⁺), using chemistry similar to that described in Example 1, step 2 starting from tert-butyl 2-[[2-[(3RS)-2,6-dioxo-3-piperidyl]-1,3-dioxo-isoindolin-4-yl]amino]acetate (Example 5, step 1) by using TFA instead of HCl.

### Step 3: N-[1-[2-[[2-[(3RS)-2,6-Dioxo-3-piperidyl]-1,3-dioxo-isoindolin-4-yl]amino]acetyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide

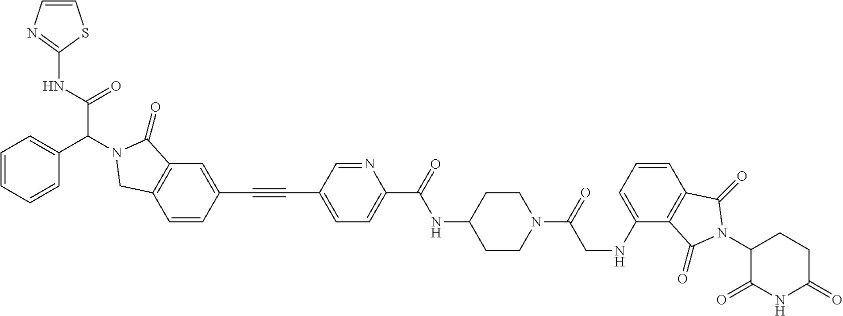 [SMILES: O=C1CCC(N2C(=O)c3cccc(NCC(=O)N4CCC(NC(=O)c5ccc(C#Cc6ccc7c(c6)C(=O)N(C(C(=O)Nc6nccs6)c6ccccc6)C7)cn5)CC4)c3C2=O)C(=O)N1]

The title compound was obtained as an orange semisolid, MS: m/e=890.5 (M+H⁺), using chemistry similar to that described in Example 1, step 1 starting from 5-[2-[3-oxo-2-[(RS)-2-oxo-1-phenyl-2-(thiazol-2-ylamino)ethyl]isoindolin-5-yl]ethynyl]-N-(4-piperidyl)pyridine-2-carboxamide hydrochloride (Example 2, step 4) and 2-[[2-[(3RS)-2,6-dioxo-3-piperidyl]-1,3-dioxo-isoindolin-4-yl]amino]acetic acid (Example 5, step 2).

### Example 6

### N-[1-[4-[[2-[(3RS)-2,6-Dioxo-3-piperidyl]-1,3-dioxo-isoindolin-4-yl]amino]butyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide

### Step 1: 2-[(3RS)-2,6-Dioxo-3-piperidyl]-4-(4-hydroxybutylamino)isoindoline-1,3-dione

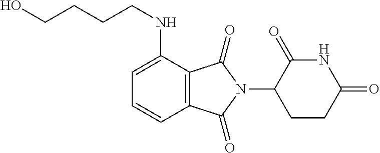 [SMILES: O=C1CCC(N2C(=O)c3cccc(NCCCCO)c3C2=O)C(=O)N1]

The title compound was obtained as an orange oil, MS: m/e=346.2 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 4-aminobutan-1-ol by using NMP instead of DMSO as solvent.

### Step 2: 4-(4-Bromobutylamino)-2-[(3RS)-2,6-dioxo-3-piperidyl]isoindoline-1,3-dione

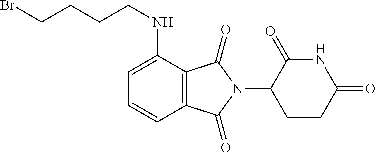 [SMILES: O=C1CCC(N2C(=O)c3cccc(NCCCCBr)c3C2=O)C(=O)N1]

A mixture of 2-[(3RS)-2,6-dioxo-3-piperidyl]-4-(4-hydroxybutylamino)isoindoline-1,3-dione (Example 6, step 1) (1 g, 2.9 mmol), triphenylphosphine (910 mg, 3.47 mmol, 1.2 equiv.) and carbon tetrabromide (1.15 g, 3.47 mmol, 1.2 equiv.) in DCM (30 ml) was stirred at room temperature for 2 hours. The mixture was evaporated and the crude product was purified by flash chromatography on a silica gel column eluting with a heptane:ethyl acetate 100:0 to 50:50 gradient to obtain the desired 4-(4-bromobutylamino)-2-[(3RS)-2,6-dioxo-3-piperidyl]isoindoline-1,3-dione (860 mg, 68% yield) as dark green foam, MS: m/e=410.2/412.2 (M+H⁺).

### Step 3: N-[1-[4-[[2-[(3RS)-2,6-Dioxo-3-piperidyl]-1,3-dioxo-isoindolin-4-yl]amino]butyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide

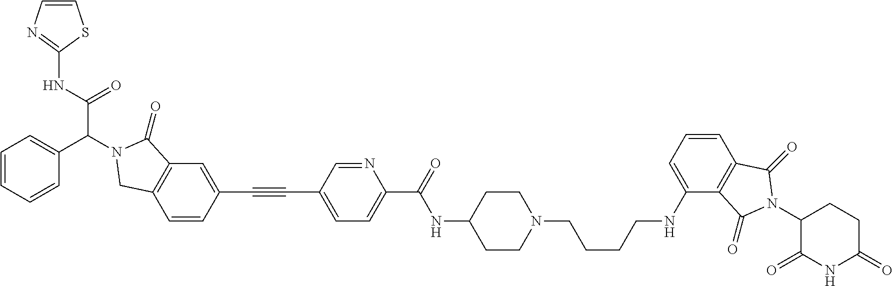 [SMILES: O=C1CCC(N2C(=O)c3cccc(NCCCCN4CCC(NC(=O)c5ccc(C#Cc6ccc7c(c6)C(=O)N(C(C(=O)Nc6nccs6)c6ccccc6)C7)cn5)CC4)c3C2=O)C(=O)N1]

5-[2-[3-Oxo-2-[(1RS)-2-oxo-1-phenyl-2-(thiazol-2-ylamino)ethyl]isoindolin-5-yl]ethynyl]-N-(4-piperidyl)pyridine-2-carboxamide hydrochloride (Example 2, step 4) (65 mg, 0.106 mmol) was dissolved in 5 ml of DMF. 4-(4-Bromobutylamino)-2-[(3RS)-2,6-dioxo-3-piperidyl]isoindoline-1,3-dione (Example 6, step 2) (52 mg, 0.127 mmol, 1.2 equiv.) and Hunig's base (82 mg, 0.636 mmol, 6 equiv.) were added at room temperature. The mixture was stirred at 60° C. for 48 hours. The reaction mixture was extracted with water and several times with dichlormethane:methanol 9:1 mixture. The organic layers were dried over sodium sulfate and evaporated to dryness. The crude product was purified by flash chromatography on a silica gel column eluting with a dichloromethane:methanol 100:0 to 90:10 gradient to obtain the desired N-[1-[4-[[2-[(3RS)-2,6-dioxo-3-piperidyl]-1,3-dioxo-isoindolin-4-yl]amino]butyl]-4-piperidyl]-5-[2-[3-oxo-2-[(1RS)-2-oxo-1-phenyl-2-(thiazol-2-ylamino)ethyl]isoindolin-5-yl]ethynyl]pyridine-2-carboxamide (12 mg, 13% yield) as a yellow semisolid, MS: m/e=904.5 (M+H⁺).

### Example 7

### N-[1-[4-[[2-[(3RS)-2,6-Dioxo-3-piperidyl]-1,3-dioxo-isoindolin-4-yl]amino]butanoyl]-4-piperidyl]-5-[2-[2-[(1RS)-1-(5-fluoro-2-hydroxy-phenyl)-2-oxo-2-(thiazol-2-ylamino)ethyl]-3-oxo-isoindolin-5-yl]ethynyl]pyridine-2-carboxamide

### Step 1: tert-Butyl [(1RS)-1-(5-fluoro-2-methoxyphenyl)-2-oxo-2-(thiazol-2-ylamino)ethyl]carbamate

The title compound was obtained as a white solid, MS: m/e=382.5 (M+H⁺), using chemistry similar to that described in Example 1, step 1 starting from (2RS)-2-((tert-butoxycarbonyl)amino)-2-(5-fluoro-2-methoxyphenyl)acetic acid.

### Step 2: (2RS)-2-Amino-2-(5-fluoro-2-methoxyphenyl)-N-(thiazol-2-yl)acetamide hydrochloride

The title compound was obtained as a light green solid, MS: m/e=282.4 (M+H⁺), using chemistry similar to that described in Example 1, step 2 starting from tert-butyl [(RS)-1-(5-fluoro-2-methoxyphenyl)-2-oxo-2-(thiazol-2-ylamino)ethyl]carbamate (Example 7, step 1).

### Step 3: (2RS)-2-(5-Fluoro-2-methoxyphenyl)-2-(6-iodo-1-oxoisoindolin-2-yl)-N-(thiazol-2-yl)acetamide

The title compound was obtained as a white solid, MS: m/e=524.4 (M+H⁺), using chemistry similar to that described in Example 1, step 3 starting from (2RS)-2-amino-2-(5-fluoro-2-methoxyphenyl)-N-(thiazol-2-yl)acetamide hydrochloride (Example 7, step 2) and methyl 2-(bromomethyl)-5-iodobenzoate.

### Step 4: Methyl 5-[2-[2-[(1RS)-1-(5-fluoro-2-methoxy-phenyl)-2-oxo-2-(thiazol-2-ylamino)ethyl]-3-oxo-isoindolin-5-yl]ethynyl]pyridine-2-carboxylate

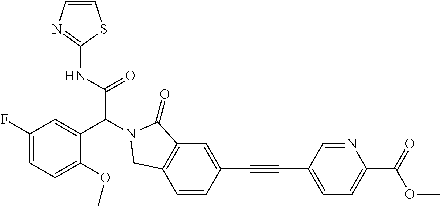 [SMILES: COC(=O)c1ccc(C#Cc2ccc3c(c2)C(=O)N(C(C(=O)Nc2nccs2)c2cc(F)ccc2OC)C3)cn1]

The title compound was obtained as a yellow solid, MS: m/e=557.3 (M+H⁺), using chemistry similar to that described in Example 1, step 5 starting from (2RS)-2-(5-fluoro-2-methoxyphenyl)-2-(6-iodo-1-oxoisoindolin-2-yl)-N-(thiazol-2-yl)acetamide (Example 7, step 3) and methyl 5-ethynylpicolinate.

### Step 5: 5-[2-[2-[(1RS)-1-(5-Fluoro-2-methoxy-phenyl)-2-oxo-2-(thiazol-2-ylamino)ethyl]-3-oxo-isoindolin-5-yl]ethynyl]pyridine-2-carboxylic acid

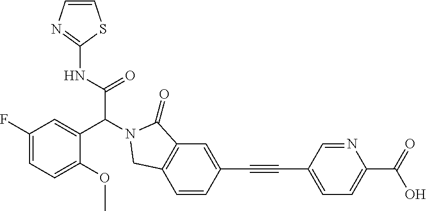 [SMILES: COc1ccc(F)cc1C(C(=O)Nc1nccs1)N1Cc2ccc(C#Cc3ccc(C(=O)O)nc3)cc2C1=O]

The title compound was obtained as a yellow solid, MS: m/e=543.3 (M+H⁺), using chemistry similar to that described in Example 2, step 2 starting from methyl 5-[2-[2-[(RS)-1-(5-fluoro-2-methoxy-phenyl)-2-oxo-2-(thiazol-2-ylamino)ethyl]-3-oxo-isoindolin-5-yl]ethynyl]pyridine-2-carboxylate (Example 7, step 4) and tert-butyl 4-aminopiperidine-1-carboxylate.

### Step 6: tert-Butyl 4-[[5-[2-[2-[(1RS)-1-(5-fluoro-2-methoxy-phenyl)-2-oxo-2-(thiazol-2-ylamino)ethyl]-3-oxo-isoindolin-5-yl]ethynyl]pyridine-2-carbonyl]amino]piperidine-1-carboxylate

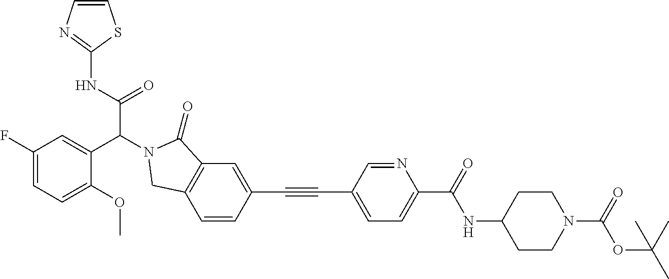 [SMILES: COc1ccc(F)cc1C(C(=O)Nc1nccs1)N1Cc2ccc(C#Cc3ccc(C(=O)NC4CCN(C(=O)OC(C)(C)C)CC4)nc3)cc2C1=O]

The title compound was obtained as a light yellow solid, MS: m/e=725.5 (M+H⁺), using chemistry similar to that described in Example 1, step 1 starting from 5-[2-[2-[(RS)-1-(5-fluoro-2-methoxy-phenyl)-2-oxo-2-(thiazol-2-ylamino)ethyl]-3-oxo-isoindolin-5-yl]ethynyl]pyridine-2-carboxylic acid (Example 7, step 5) and tert-butyl 4-aminopiperidine-1-carboxylate.

### Step 7: 5-[2-[2-[(1RS)-1-(5-Fluoro-2-methoxy-phenyl)-2-oxo-2-(thiazol-2-ylamino)ethyl]-3-oxo-isoindolin-5-yl]ethynyl]-N-(4-piperidyl)pyridine-2-carboxamide hydrochloride

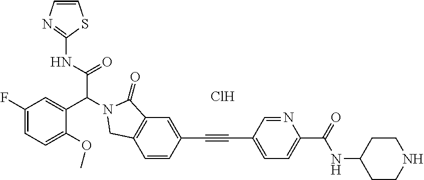 [SMILES: COc1ccc(F)cc1C(C(=O)Nc1nccs1)N1Cc2ccc(C#Cc3ccc(C(=O)NC4CCNCC4)nc3)cc2C1=O ; Cl]

The title compound was obtained as a yellow solid, MS: m/e=625.4 (M+H⁺), using chemistry similar to that described in Example 1, step 2 starting from tert-butyl 4-[[5-[2-[2-[(1RS)-1-(5-fluoro-2-methoxy-phenyl)-2-oxo-2-(thiazol-2-ylamino)ethyl]-3-oxo-isoindolin-5-yl]ethynyl]pyridine-2-carbonyl]amino]piperidine-1-carboxylate (Example 7, step 6).

### Step 8: N-[1-[4-[[2-[(3RS)-2,6-Dioxo-3-piperidyl]-1,3-dioxo-isoindolin-4-yl]amino]butanoyl]-4-piperidyl]-5-[2-[2-[(1RS)-1-(5-fluoro-2-methoxy-phenyl)-2-oxo-2-(thiazol-2-ylamino)ethyl]-3-oxo-isoindolin-5-yl]ethynyl]pyridine-2-carboxamide

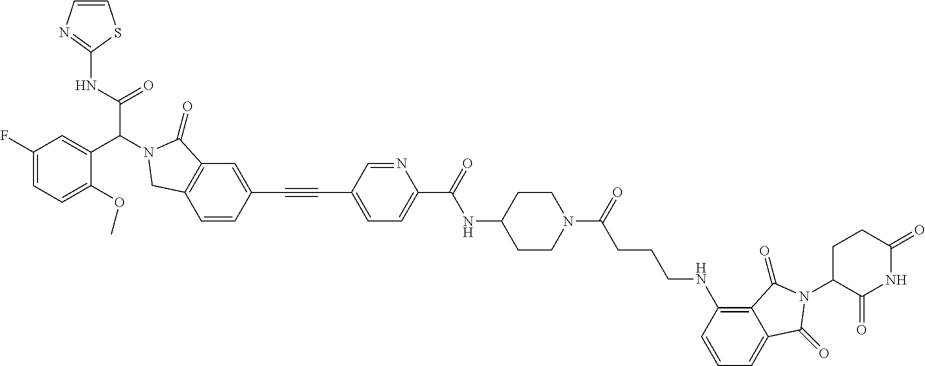 [SMILES: COc1ccc(F)cc1C(C(=O)Nc1nccs1)N1Cc2ccc(C#Cc3ccc(C(=O)NC4CCN(C(=O)CCCNc5cccc6c5C(=O)N(C5CCC(=O)NC5=O)C6=O)CC4)nc3)cc2C1=O]

The title compound was obtained as a yellow solid, MS: m/e=966.7 (M+H⁺), using chemistry similar to that described in Example 1, step 1 starting from 5-[2-[2-[(RS)-1-(5-fluoro-2-methoxy-phenyl)-2-oxo-2-(thiazol-2-ylamino)ethyl]-3-oxo-isoindolin-5-yl]ethynyl]-N-(4-piperidyl)pyridine-2-carboxamide hydrochloride (Example 7, step 7) and 4-[[2-[(3RS)-2,6-dioxo-3-piperidyl]-1,3-dioxo-isoindolin-4-yl]amino]butanoic acid (Example 2, step 5).

### Step 9: N-[1-[4-[[2-[(3RS)-2,6-Dioxo-3-piperidyl]-1,3-dioxo-isoindolin-4-yl]amino]butanoyl]-4-piperidyl]-5-[2-[2-[(1RS)-1-(5-fluoro-2-hydroxy-phenyl)-2-oxo-2-(thiazol-2-ylamino)ethyl]-3-oxo-isoindolin-5-yl]ethynyl]pyridine-2-carboxamide

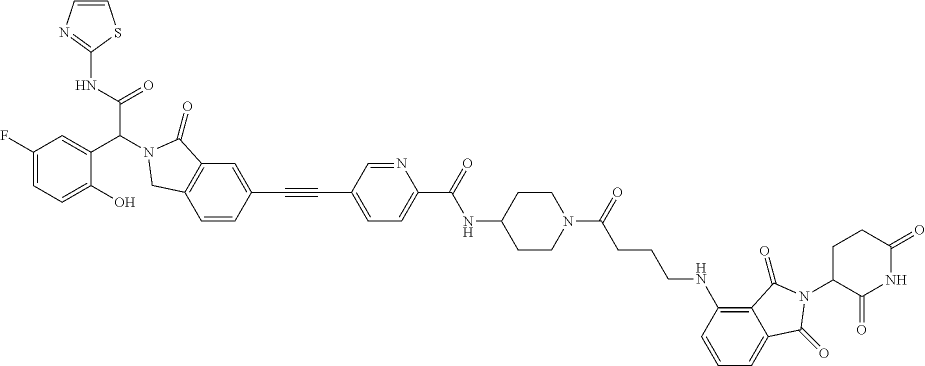 [SMILES: O=C1CCC(N2C(=O)c3cccc(NCCCC(=O)N4CCC(NC(=O)c5ccc(C#Cc6ccc7c(c6)C(=O)N(C(C(=O)Nc6nccs6)c6cc(F)ccc6O)C7)cn5)CC4)c3C2=O)C(=O)N1]

N-[1-[4-[[2-[(3RS)-2,6-Dioxo-3-piperidyl]-1,3-dioxo-isoindolin-4-yl]amino]butanoyl]-4-piperidyl]-5-[2-[2-[(1RS)-1-(5-fluoro-2-methoxy-phenyl)-2-oxo-2-(thiazol-2-ylamino)ethyl]-3-oxo-isoindolin-5-yl]ethynyl]pyridine-2-carboxamide (Example 7, step 8) (40 mg, 0.041 mmol) was dissolved in 1 ml of dichloromethane and cooled to 0-5° C. BBr3 (1M in dichloromethane) (0.16 ml, 0.16 mmol, 4 equiv.) was added drop wise and the mixture stirred for 1 hour at room temperature. The mixture was cooled to 0-5° C. and water (45 μl, 2.48 mmol, 60 equiv.) was added drop wise. The mixture was stirred for 10 minutes and evaporated with Isolute® to dryness. The crude product was purified by flash chromatography on a silica gel column eluting with a methanol:dichloromethane 0:100 to 20:80 gradient. The desired N-[1-[4-[[2-[(3RS)-2,6-dioxo-3-piperidyl]-1,3-dioxo-isoindolin-4-yl]amino]butanoyl]-4-piperidyl]-5-[2-[2-[(1RS)-1-(5-fluoro-2-hydroxy-phenyl)-2-oxo-2-(thiazol-2-ylamino)ethyl]-3-oxo-isoindolin-5-yl]ethynyl]pyridine-2-carboxamide (22 mg, 55% yield) was obtained as a yellow solid, MS: m/e=952.8 (M+H⁺).

### Example 8

### N-[1-[4-[[2-[(3RS)-2,6-Dioxo-3-piperidyl]-1,3-dioxo-isoindolin-4-yl]amino]butyl]-4-piperidyl]-3-fluoro-5-[2-[3-oxo-2-[(1RS)-2-oxo-1-phenyl-2-(thiazol-2-ylamino)ethyl]isoindolin-5-yl]ethynyl]pyridine-2-carboxamide

### Step 1: (2RS)-2-[1-Oxo-6-(2-trimethylsilylethynyl)isoindolin-2-yl]-2-phenyl-N-thiazol-2-yl-acetamide

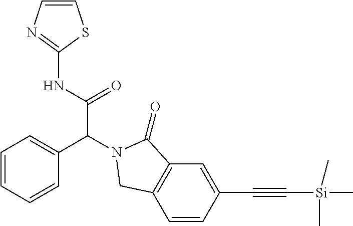 [SMILES: C[Si](C)(C)C#Cc1ccc2c(c1)C(=O)N(C(C(=O)Nc1nccs1)c1ccccc1)C2]

The title compound was obtained as a light yellow foam, MS: m/e=446.3 (M+H⁺), using chemistry similar to that described in Example 1, step 5 starting from (2RS)-2-(6-iodo-1-oxo-isoindolin-2-yl)-2-phenyl-N-thiazol-2-yl-acetamide (Example 1, step 3) and ethynyltrimethylsilane.

### Step 2: Methyl 3-fluoro-5-[2-[3-oxo-2-[(1RS)-2-oxo-1-phenyl-2-(thiazol-2-ylamino)ethyl]isoindolin-5-yl]ethynyl]pyridine-2-carboxylate

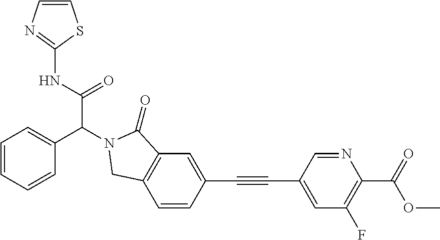 [SMILES: COC(=O)c1ncc(C#Cc2ccc3c(c2)C(=O)N(C(C(=O)Nc2nccs2)c2ccccc2)C3)cc1F]

The title compound was obtained as a yellow solid, MS: m/e=527.3 (M+H⁺), using chemistry similar to that described in Example 1, step 5 starting from (2RS)-2-[1-oxo-6-(2-trimethylsilylethynyl)isoindolin-2-yl]-2-phenyl-N-thiazol-2-yl-acetamide (Example 8, step 1) and methyl 5-bromo-3-fluoropicolinate by using TBAF for the cleavage of the trimethylsilyl protecting group.

### Step 3: 3-Fluoro-5-[2-[3-oxo-2-[(1RS)-2-oxo-1-phenyl-2-(thiazol-2-ylamino)ethyl]isoindolin-5-yl]ethynyl]pyridine-2-carboxylic acid

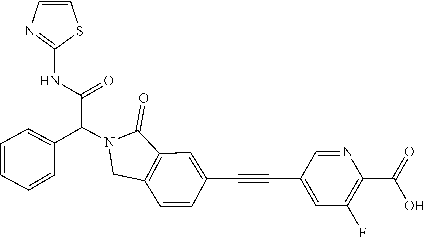 [SMILES: O=C(O)c1ncc(C#Cc2ccc3c(c2)C(=O)N(C(C(=O)Nc2nccs2)c2ccccc2)C3)cc1F]

The title compound was obtained as a yellow solid, MS: m/e=513.3 (M+H⁺), using chemistry similar to that described in Example 2, step 2 starting from methyl 3-fluoro-5-[2-[3-oxo-2-[(1RS)-2-oxo-1-phenyl-2-(thiazol-2-ylamino)ethyl]isoindolin-5-yl]ethynyl]pyridine-2-carboxylate (Example 8, step 2).

### Step 4: tert-Butyl 4-[[3-fluoro-5-[2-[3-oxo-2-[(1RS)-2-oxo-1-phenyl-2-(thiazol-2-ylamino)ethyl]isoindolin-5-yl]ethynyl]pyridine-2-carbonyl]amino]piperidine-1-carboxylate

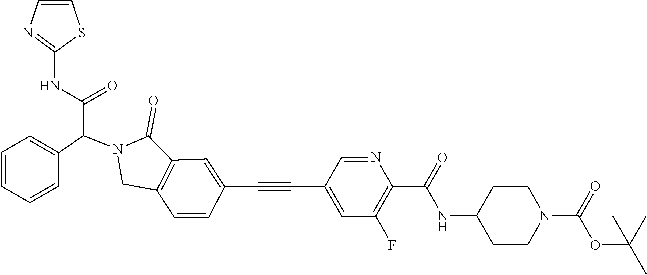 [SMILES: CC(C)(C)OC(=O)N1CCC(NC(=O)c2ncc(C#Cc3ccc4c(c3)C(=O)N(C(C(=O)Nc3nccs3)c3ccccc3)C4)cc2F)CC1]

The title compound was obtained as an orange foam, MS: m/e=695.6 (M+H⁺), using chemistry similar to that described in Example 1, step 1 starting from 3-fluoro-5-[2-[3-oxo-2-[(1RS)-2-oxo-1-phenyl-2-(thiazol-2-ylamino)ethyl]isoindolin-5-yl]ethynyl]pyridine-2-carboxylic acid (Example 8, step 3) and tert-butyl 4-aminopiperidine-1-carboxylate.

### Step 4: 3-Fluoro-5-[2-[3-oxo-2-[(1RS)-2-oxo-1-phenyl-2-(thiazol-2-ylamino)ethyl]isoindolin-5-yl]ethynyl]-N-(4-piperidyl)pyridine-2-carboxamide hydrochloride

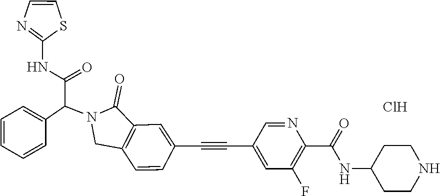 [SMILES: O=C(NC1CCNCC1)c1ncc(C#Cc2ccc3c(c2)C(=O)N(C(C(=O)Nc2nccs2)c2ccccc2)C3)cc1F ; Cl]

The title compound was obtained as a yellow solid, MS: m/e=595.4 (M+H⁺), using chemistry similar to that described in Example 1, step 2 starting from tert-butyl 4-[[3-fluoro-5-[2-[3-oxo-2-[(1RS)-2-oxo-1-phenyl-2-(thiazol-2-ylamino)ethyl]isoindolin-5-yl]ethynyl]pyridine-2-carbonyl]amino]piperidine-1-carboxylate (Example 8, step 4).

### Step 5: N-[1-[4-[[2-[(3RS)-2,6-Dioxo-3-piperidyl]-1,3-dioxo-isoindolin-4-yl]amino]butyl]-4-piperidyl]-3-fluoro-5-[2-[3-oxo-2-[(1RS)-2-oxo-1-phenyl-2-(thiazol-2-ylamino)ethyl]isoindolin-5-yl]ethynyl]pyridine-2-carboxamide

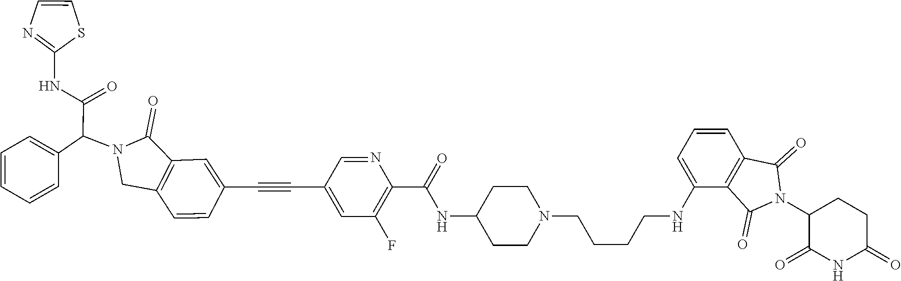 [SMILES: O=C1CCC(N2C(=O)c3cccc(NCCCCN4CCC(NC(=O)c5ncc(C#Cc6ccc7c(c6)C(=O)N(C(C(=O)Nc6nccs6)c6ccccc6)C7)cc5F)CC4)c3C2=O)C(=O)N1]

The title compound was obtained as a yellow solid, MS: m/e=922.5 (M+H⁺), using chemistry similar to that described in Example 6, step 3 starting from 3-fluoro-5-[2-[3-oxo-2-[(1RS)-2-oxo-1-phenyl-2-(thiazol-2-ylamino)ethyl]isoindolin-5-yl]ethynyl]-N-(4-piperidyl)pyridine-2-carboxamide hydrochloride (Example 8, step 4) and 4-(4-bromobutylamino)-2-[(3RS)-2,6-dioxo-3-piperidyl]isoindoline-1,3-dione (Example 6, step 2).

### Example 9

### N-[1-[4-[[2-[(3RS)-2,6-Dioxo-3-piperidyl]-1,3-dioxo-isoindolin-4-yl]amino]butyl]-4-piperidyl]-5-[2-[3-oxo-2-[(1RS)-2-oxo-1-phenyl-2-(2-pyridylamino)ethyl]isoindolin-5-yl]ethynyl]pyridine-2-carboxamide

### Step 1: (2RS)-2-(6-Bromo-1-oxo-isoindolin-2-yl)-2-phenyl-acetic acid

6-Bromoisoindolin-1-one (4 g, 18.9 mmol) was suspended in 70 ml of THF and cooled to 0-5° C. Sodium hydride (60% in mineral oil) (1.5 g, 37.7 mmol, 2 equiv.) was added in portions at 0-5° C. and after 5 minutes (2RS)-2-bromo-2-phenyl-acetic acid (4.34 g, 20.2 mmol, 1.07 equiv.) were added and the mixture was stirred at 0-5° C. for 2 hours. The reaction mixture was extracted with 1M HCl solution and twice with ethyl acetate. The organic layers were dried over sodium sulfate and evaporated to dryness to obtain the desired (2RS)-2-(6-bromo-1-oxo-isoindolin-2-yl)-2-phenyl-acetic acid (5.78 g, 89% yield) as a white solid, MS: m/e=345.9/347.9 (M+H⁺).

### Step 2: (2RS)-2-(6-Bromo-1-oxo-isoindolin-2-yl)-2-phenyl-N-(2-pyridyl)acetamide

The title compound was obtained as a light yellow solid, MS: m/e=421.9/423.9 (M+H⁺), using chemistry similar to that described in Example 1, step 1 starting from (2RS)-2-(6-bromo-1-oxo-isoindolin-2-yl)-2-phenyl-acetic acid (Example 9, step 1) and 2-aminopyridine.

### Step 3: Methyl 5-[2-[3-oxo-2-[(1RS)-2-oxo-1-phenyl-2-(2-pyridylamino)ethyl]isoindolin-5-yl]ethynyl]pyridine-2-carboxylate

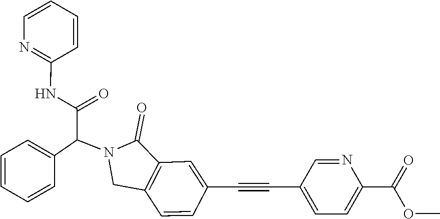 [SMILES: COC(=O)c1ccc(C#Cc2ccc3c(c2)C(=O)N(C(C(=O)Nc2ccccn2)c2ccccc2)C3)cn1]

The title compound was obtained as a yellow solid, MS: m/e=503.4 (M+H⁺), using chemistry similar to that described in Example 1, step 5 starting from (2RS)-2-(6-bromo-1-oxo-isoindolin-2-yl)-2-phenyl-N-(2-pyridyl)acetamide (Example 9, step 2) and methyl 5-ethynylpicolinate.

### Step 4: 5-[2-[3-Oxo-2-[(1RS)-2-oxo-1-phenyl-2-(2-pyridylamino)ethyl]isoindolin-5-yl]ethynyl]pyridine-2-carboxylic acid

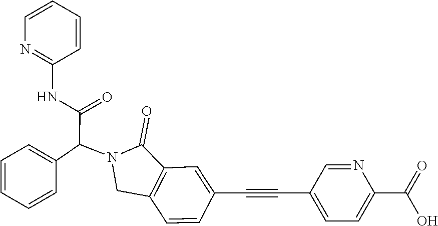 [SMILES: O=C(O)c1ccc(C#Cc2ccc3c(c2)C(=O)N(C(C(=O)Nc2ccccn2)c2ccccc2)C3)cn1]

The title compound was obtained as a white solid, MS: m/e=489.4 (M+H⁺), using chemistry similar to that described in Example 2, step 2 starting from methyl 5-[2-[3-oxo-2-[(RS)-2-oxo-1-phenyl-2-(2-pyridylamino)ethyl]isoindolin-5-yl]ethynyl]pyridine-2-carboxylate (Example 9, step 3) and tert-butyl 4-aminopiperidine-1-carboxylate.

### Step 5: tert-Butyl 4-[[5-[2-[3-oxo-2-[(1RS)-2-oxo-1-phenyl-2-(2-pyridylamino)ethyl]isoindolin-5-yl]ethynyl]pyridine-2-carbonyl]amino]piperidine-1-carboxylate

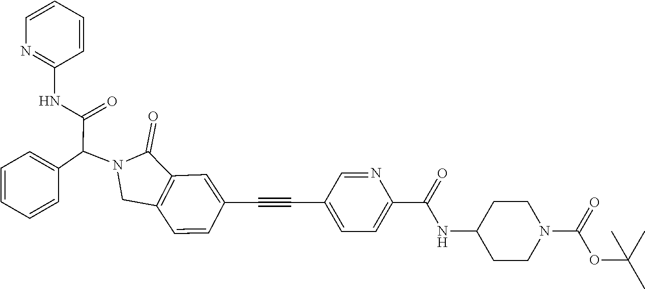 [SMILES: CC(C)(C)OC(=O)N1CCC(NC(=O)c2ccc(C#Cc3ccc4c(c3)C(=O)N(C(C(=O)Nc3ccccn3)c3ccccc3)C4)cn2)CC1]

The title compound was obtained as a white foam, MS: m/e=671.6 (M+H⁺), using chemistry similar to that described in Example 1, step 1 starting from 5-[2-[3-oxo-2-[(RS)-2-oxo-1-phenyl-2-(2-pyridylamino)ethyl]isoindolin-5-yl]ethynyl]pyridine-2-carboxylic acid (Example 9, step 4) and tert-butyl 4-aminopiperidine-1-carboxylate.

### Step 6: 5-[2-[3-Oxo-2-[(1RS)-2-oxo-1-phenyl-2-(2-pyridylamino)ethyl]isoindolin-5-yl]ethynyl]-N-(4-piperidyl)pyridine-2-carboxamide

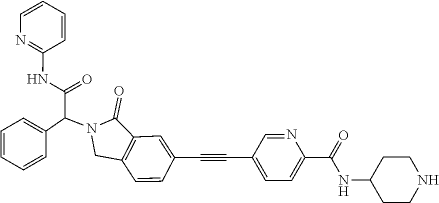 [SMILES: O=C(NC1CCNCC1)c1ccc(C#Cc2ccc3c(c2)C(=O)N(C(C(=O)Nc2ccccn2)c2ccccc2)C3)cn1]

The title compound was obtained as a white solid, MS: m/e=571.5 (M+H⁺), using chemistry similar to that described in Example 1, step 2 starting from tert-butyl 4-[[5-[2-[3-oxo-2-[(1RS)-2-oxo-1-phenyl-2-(2-pyridylamino)ethyl]isoindolin-5-yl]ethynyl]pyridine-2-carbonyl]amino]piperidine-1-carboxylate (Example 9, step 5).

### Step 7: N-[1-[4-[[2-[(3RS)-2,6-Dioxo-3-piperidyl]-1,3-dioxo-isoindolin-4-yl]amino]butyl]-4-piperidyl]-5-[2-[3-oxo-2-[(1RS)-2-oxo-1-phenyl-2-(2-pyridylamino)ethyl]isoindolin-5-yl]ethynyl]pyridine-2-carboxamide

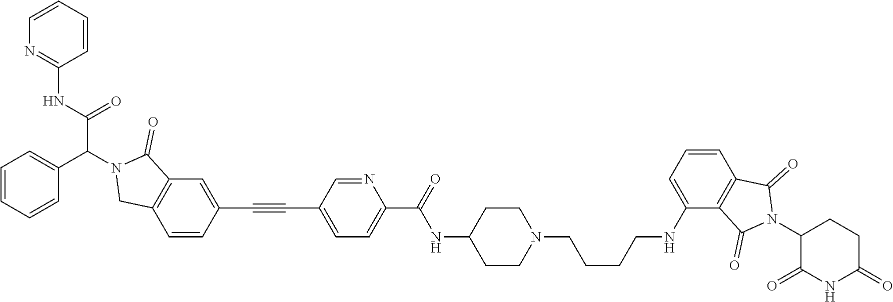 [SMILES: O=C1CCC(N2C(=O)c3cccc(NCCCCN4CCC(NC(=O)c5ccc(C#Cc6ccc7c(c6)C(=O)N(C(C(=O)Nc6ccccn6)c6ccccc6)C7)cn5)CC4)c3C2=O)C(=O)N1]

The title compound was obtained as a yellow foam, MS: m/e=899.0 (M+H⁺), using chemistry similar to that described in Example 6, step 3 starting from 5-[2-[3-oxo-2-[(RS)-2-oxo-1-phenyl-2-(2-pyridylamino)ethyl]isoindolin-5-yl]ethynyl]-N-(4-piperidyl)pyridine-2-carboxamide (Example 9, step 6) and 4-(4-bromobutylamino)-2-[(3RS)-2,6-dioxo-3-piperidyl]isoindoline-1,3-dione (Example 6, step 2).

### Example 10

### N-[1-[4-[[2-[(3RS)-2,6-Dioxo-3-piperidyl]-1,3-dioxo-isoindolin-4-yl]amino]butyl]-4-piperidyl]-5-[2-[2-[(1RS)-1-(5-fluoro-2-hydroxy-phenyl)-2-oxo-2-(thiazol-2-ylamino)ethyl]-3-oxo-isoindolin-5-yl]ethynyl]pyridine-2-carboxamide

The title compound was obtained as a yellow solid, MS: m/e=938.9 (M+H⁺), using chemistry similar to that described in Example 6, step 3 and Example 7, step 9 starting from 5-[2-[2-[(1RS)-1-(5-fluoro-2-methoxy-phenyl)-2-oxo-2-(thiazol-2-ylamino)ethyl]-3-oxo-isoindolin-5-yl]ethynyl]-N-(4-piperidyl)pyridine-2-carboxamide hydrochloride (Example 7, step 7) and 4-(4-bromobutylamino)-2-[(3RS)-2,6-dioxo-3-piperidyl]isoindoline-1,3-dione (Example 6, step 2).

### Example 11

### N-[1-[[1-[2-[(3RS)-2,6-Dioxo-3-piperidyl]-1,3-dioxo-isoindolin-5-yl]-4-piperidyl]methyl]-4-piperidyl]-5-[2-[3-oxo-2-[(1RS)-2-oxo-1-phenyl-2-(2-pyridylamino)ethyl]isoindolin-5-yl]ethynyl]pyridine-2-carboxamide

### Step 1: 5-[4-(Bromomethyl)-1-piperidyl]-2-[(3RS)-2,6-dioxo-3-piperidyl]isoindoline-1,3-dione

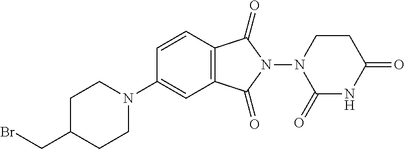 [SMILES: O=C1CCN(N2C(=O)c3ccc(N4CCC(CBr)CC4)cc3C2=O)C(=O)N1]

The title compound was obtained as a yellow solid, MS: m/e=434.0/436.0 (M+H⁺), using chemistry similar to that described in Example 6, step 1 and step 2 starting from 2-[(3RS)-2,6-dioxo-3-piperidyl]-5-fluoro-isoindoline-1,3-dione (CAS 835616-61-0) and 4-piperidylmethanol.

### Step 2: N-[1-[[1-[2-[(3RS)-2,6-Dioxo-3-piperidyl]-1,3-dioxo-isoindolin-5-yl]-4-piperidyl]methyl]-4-piperidyl]-5-[2-[3-oxo-2-[(1RS)-2-oxo-1-phenyl-2-(2-pyridylamino)ethyl]isoindolin-5-yl]ethynyl]pyridine-2-carboxamide

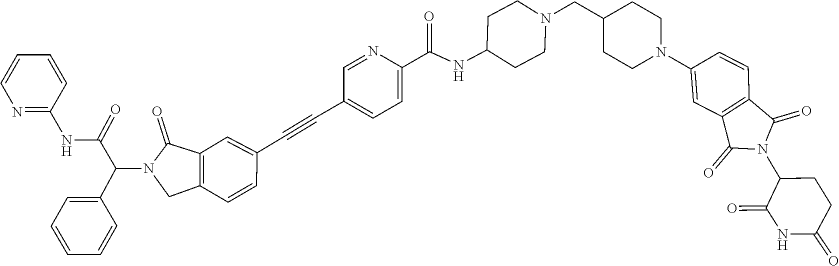 [SMILES: O=C1CCC(N2C(=O)c3ccc(N4CCC(CN5CCC(NC(=O)c6ccc(C#Cc7ccc8c(c7)C(=O)N(C(C(=O)Nc7ccccn7)c7ccccc7)C8)cn6)CC5)CC4)cc3C2=O)C(=O)N1]

The title compound was obtained as a yellow solid, MS: m/e=925.7 (M+H⁺), using chemistry similar to that described in Example 6, step 3 starting from 5-[2-[3-oxo-2-[(RS)-2-oxo-1-phenyl-2-(2-pyridylamino)ethyl]isoindolin-5-yl]ethynyl]-N-(4-piperidyl)pyridine-2-carboxamide (Example 9, step 6) and 5-[4-(bromomethyl)-1-piperidyl]-2-[(3RS)-2,6-dioxo-3-piperidyl]isoindoline-1,3-dione (Example 11, step 1).

### Example 12

### (2RS)-2-[6-[2-[6-[[4-[2-[1-[2-[(3RS)-2,6-Dioxo-3-piperidyl]-1,3-dioxo-isoindolin-4-yl]-4-piperidyl]acetyl]piperazin-1-yl]methyl]-3-pyridyl]ethynyl]-1-oxo-isoindolin-2-yl]-2-phenyl-N-(2-pyridyl)acetamide

### Step 1: (2RS)-2-[1-Oxo-6-[2-[6-(piperazin-1-ylmethyl-3-pyridyl]ethynyl]isoindolin-2-yl]-2-phenyl-N-(2-pyridyl)acetamide hydrochloride

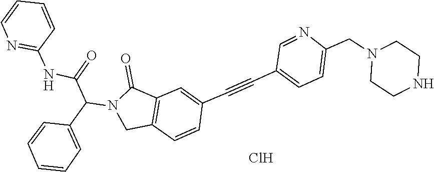 [SMILES: O=C(Nc1ccccn1)C(c1ccccc1)N1Cc2ccc(C#Cc3ccc(CN4CCNCC4)nc3)cc2C1=O ; Cl]

The title compound was obtained as a yellow solid, MS: m/e=543.4 (M+H⁺), using chemistry similar to that described in Example 1, step 5 and step 6 starting from (2RS)-2-(6-bromo-1-oxo-isoindolin-2-yl)-2-phenyl-N-(2-pyridyl)acetamide (Example 9, step 2) and tert-butyl 4-[(5-ethynyl-2-pyridyl)methyl]piperazine-1-carboxylate (Example 4, step 1).

### Step 2: 2-[1-[2-[(3RS)-2,6-Dioxo-3-piperidyl]-1,3-dioxo-isoindolin-4-yl]-4-piperidyl]acetic acid

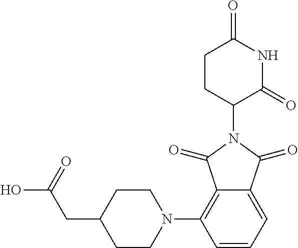 [SMILES: O=C(O)CC1CCN(c2cccc3c2C(=O)N(C2CCC(=O)NC2=O)C3=O)CC1]

The title compound was obtained as a yellow solid, MS: m/e=400.1 (M+H⁺), using chemistry similar to that described in Example 1, step 7 starting from 2-[(3RS)-2,6-dioxo-3-piperidyl]-4-fluoro-isoindoline-1,3-dione (CAS 835616-60-9) and 2-(4-piperidyl)acetic acid hydrochloride.

### Step 3: (2RS)-2-[6-[2-[6-[[4-[2-[1-[2-[(3RS)-2,6-Dioxo-3-piperidyl]-1,3-dioxo-isoindolin-4-yl]-4-piperidyl]acetyl]piperazin-1-yl]methyl]-3-pyridyl]ethynyl]-1-oxo-isoindolin-2-yl]-2-phenyl-N-(2-pyridyl)acetamide

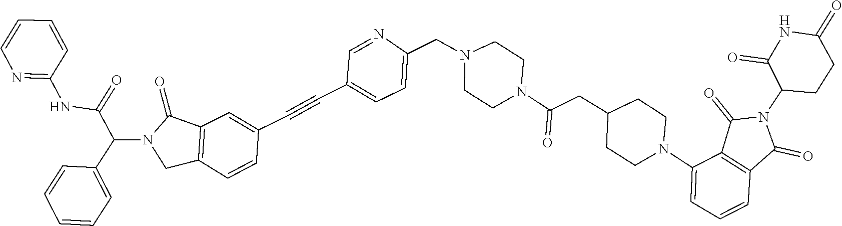 [SMILES: O=C1CCC(N2C(=O)c3cccc(N4CCC(CC(=O)N5CCN(Cc6ccc(C#Cc7ccc8c(c7)C(=O)N(C(C(=O)Nc7ccccn7)c7ccccc7)C8)cn6)CC5)CC4)c3C2=O)C(=O)N1]

The title compound was obtained as a yellow solid, MS: m/e=924.6 (M−H⁺), using chemistry similar to that described in Example 1, step 1 starting from (2RS)-2-[1-oxo-6-[2-[6-(piperazin-1-ylmethyl)-3-pyridyl]ethynyl]isoindolin-2-yl]-2-phenyl-N-(2-pyridyl)acetamide hydrochloride (example 12, step 1) and 2-[1-[2-[(3RS)-2,6-dioxo-3-piperidyl]-1,3-dioxo-isoindolin-4-yl]-4-piperidyl]acetic acid (Example 12, step 2).

We claim:

1. A compound of formula I, or a pharmaceutically acceptable salt thereof, 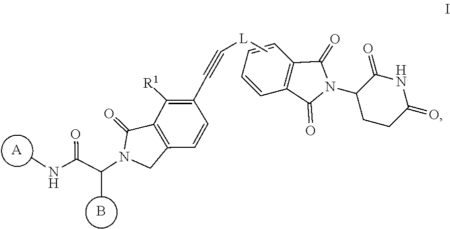
wherein
L is selected from the group consisting of
i) -aryl-(CH₂)₁₋₂-heterocyclyl-C(═O)—(CH₂)₁₋₁₀—NH—;
ii) -heteroaryl-C(═O)—NH-heterocyclyl-C(═O)—(CH₂)₁₋₁₀—NH—;
iii) -heteroaryl-(CH₂)₁₋₂-heterocyclyl-C(═O)—(CH₂)₁₋₁₀—NH—;
iv) -heteroaryl-C(═O)—NH-heterocyclyl-(CH₂)₁₋₁₀—NH—;
v) -heteroaryl-C(═O)—NH-heterocyclyl-(CH₂)₁₋₁₀-heterocyclyl-; and
vi) -heteroaryl-(CH₂)₁₋₂-heterocyclyl-C(═O)—(CH₂)₁₋₁₀-heterocyclyl-;
wherein each aryl or heteroaryl moiety can be independently substituted by
a) halogen, or
b) C₁₋₆alkyl;
R¹ is H;
A is heteroaryl
B is aryl which unsubstituted or substituted by 1-2 substituents individually selected from
a) halogen,
b) C₁₋₆alkyl, and
c) hydroxy.

2. The compound of claim 1, or a pharmaceutically acceptable salt thereof, wherein L is selected from the group consisting of
i) -5F-pyridinyl-C(═O)—NH-piperidyl-(CH₂)₄—NH—,
ii) -phenyl-(CH₂)1-piperazinyl-C(═O)—(CH₂)₃—NH—,
iii) -phenyl-(CH₂)1-piperazinyl-C(═O)—(CH₂)₅—NH—,
iv) -pyridinyl-(CH₂)1-piperazinyl-C(═O)—(CH₂)₁-piperidyl-,
v) -pyridinyl-(CH₂)1-piperazinyl-C(═O)—(CH₂)₃—NH—,
vi) -pyridinyl-C(═O)—NH-piperidyl-(CH₂)₁-piperidyl-,
vii) -pyridinyl-C(═O)—NH-piperidyl-(CH₂)₄—NH—,
viii) -pyridinyl-C(═O)—NH-piperidyl-C(═O)—(CH₂)₁—NH—, and
ix) -pyridinyl-C(═O)—NH-piperidyl-C(═O)—(CH₂)₃—NH—.

3. The compound of claim 1, or a pharmaceutically acceptable salt thereof, wherein A is thiazolyl.

4. The compound of claim 1, or a pharmaceutically acceptable salt thereof, wherein A is pyridinyl.

5. The compound of claim 1, or a pharmaceutically acceptable salt thereof, wherein B is phenyl.

6. The compound of claim 1, or a pharmaceutically acceptable salt thereof, wherein B is substituted with 1 or 2 individually selected substituents selected from F, methyl, and hydroxy.

7. The compound of claim 1, wherein L is phenyl-(CH₂)₁₋₂-piperazinyl-C(═O)—(CH₂)₁₋₁₀—NH— wherein phenyl can be independently substituted by halogen or C₁₋₆alkyl.

8. The compound of claim 1, wherein L is pyridinyl-C(═O)—NH-piperidyl-C(═O)—(CH₂)₁₋₁₀—NH— wherein pyridinyl can be independently substituted by halogen or C₁₋₆alkyl.

9. The compound of claim 1, wherein L is pyridinyl-(CH₂)₁₋₂-piperazinyl-C(═O)—(CH₂)₁₋₁₀—NH wherein pyridinyl can be independently substituted by halogen or C₁₋₆alkyl.

10. The compound of claim 1, wherein L is pyridinyl-C(═O)—NH-piperidyl-(CH₂)₁₋₁₀—NH-wherein pyridinyl can be independently substituted by halogen or C₁₋₆alkyl.

11. The compound of claim 1, wherein L is pyridinyl-C(═O)—NH-piperidyl-(CH₂)₁₋₁₀-piperidyl-wherein pyridinyl can be independently substituted by halogen or C₁₋₆alkyl.

12. The compound of claim 1, wherein L is pyridinyl-(CH₂)₁₋₂-piperidyl-C(═O)—(CH₂)₁₋₁₀-piperidyl- wherein pyridinyl can be independently substituted by halogen or C₁₋₆alkyl.

13. The compound of claim 1, wherein L is -pyridinyl-(CH₂)₁₋₂-piperazinyl-C(═O)—(CH₂)₁₋₁₀-piperidyl- wherein pyridinyl can be independently substituted by halogen or C₁₋₆alkyl.

14. The compound of claim 1, or a pharmaceutically acceptable salt thereof, selected from the group consisting of

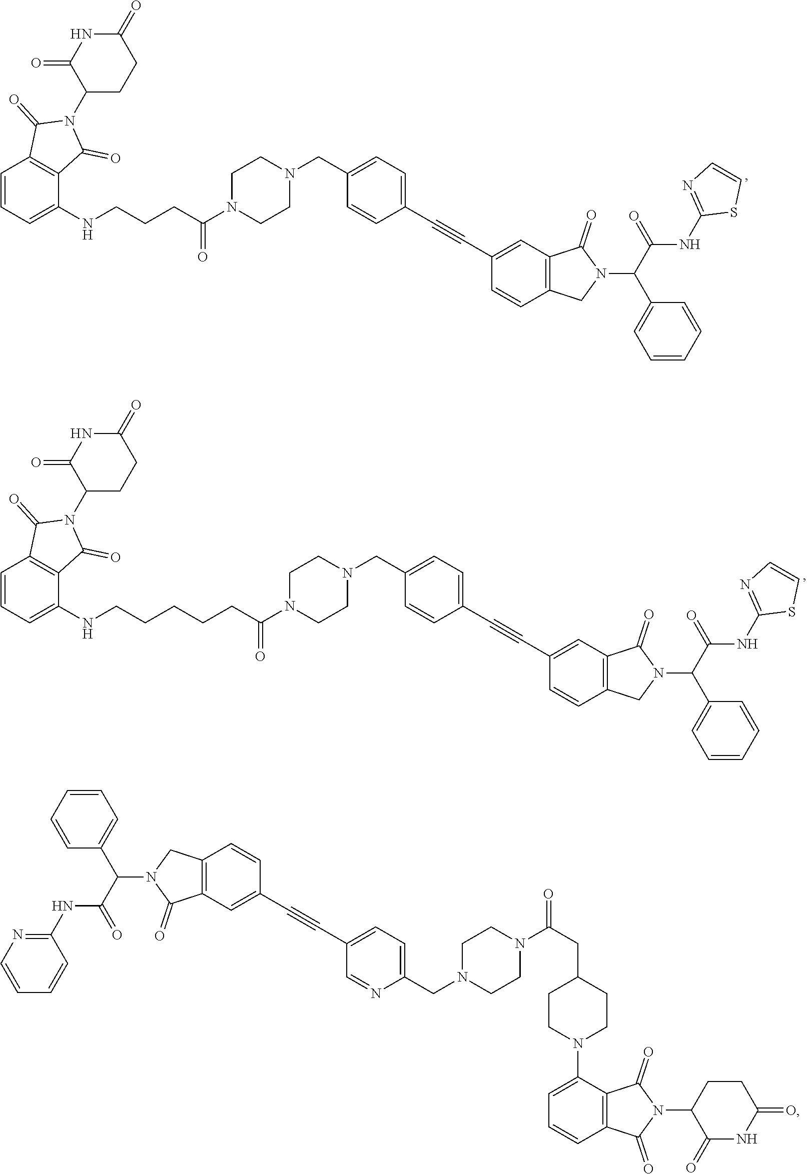 [SMILES: O=C1CCC(N2C(=O)c3cccc(NCCCC(=O)N4CCN(Cc5ccc(C#Cc6ccc7c(c6)C(=O)N(C(C(=O)Nc6nccs6)c6ccccc6)C7)cc5)CC4)c3C2=O)C(=O)N1 ; O=C1CCC(N2C(=O)c3cccc(NCCCCCC(=O)N4CCN(Cc5ccc(C#Cc6ccc7c(c6)C(=O)N(C(C(=O)Nc6nccs6)c6ccccc6)C7)cc5)CC4)c3C2=O)C(=O)N1 ; O=C1CCC(N2C(=O)c3cccc(N4CCC(CC(=O)N5CCN(Cc6ccc(C#Cc7ccc8c(c7)C(=O)N(C(C(=O)Nc7ccccn7)c7ccccc7)C8)cn6)CC5)CC4)c3C2=O)C(=O)N1]

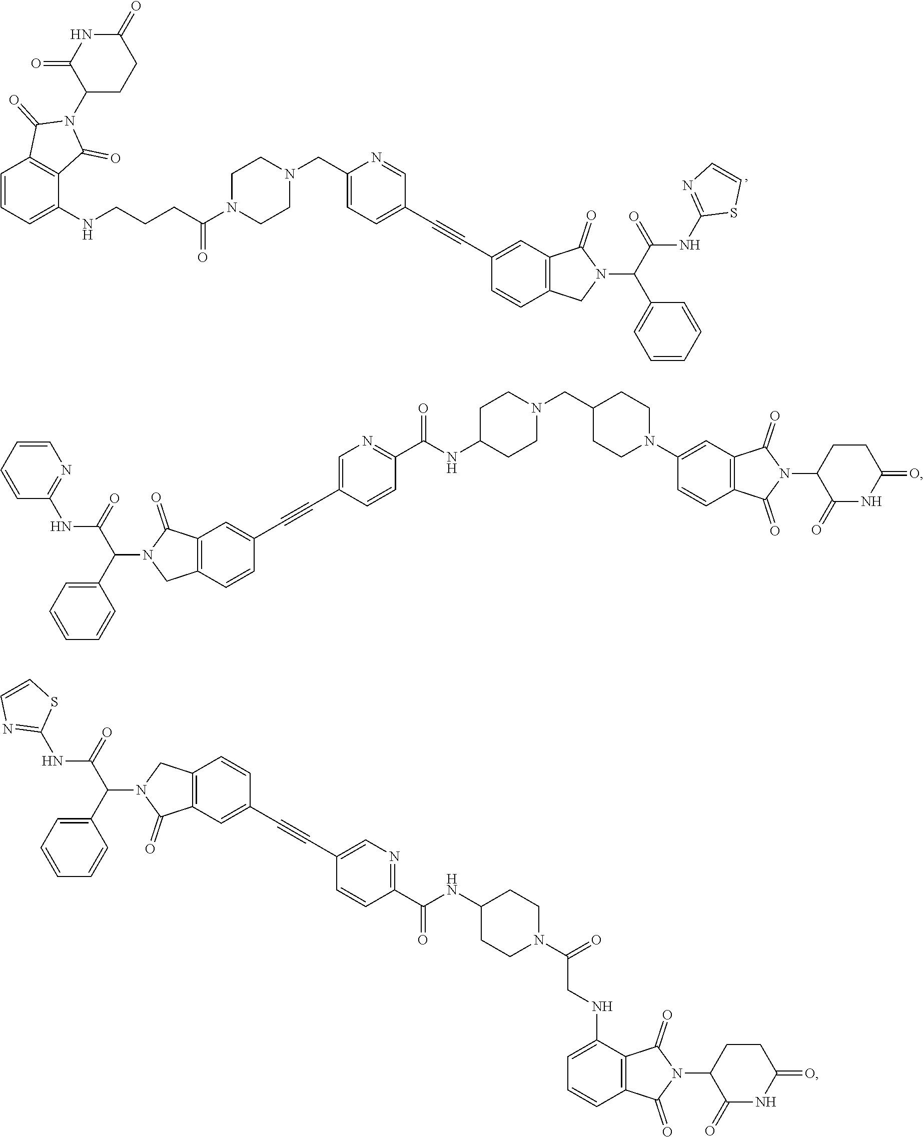

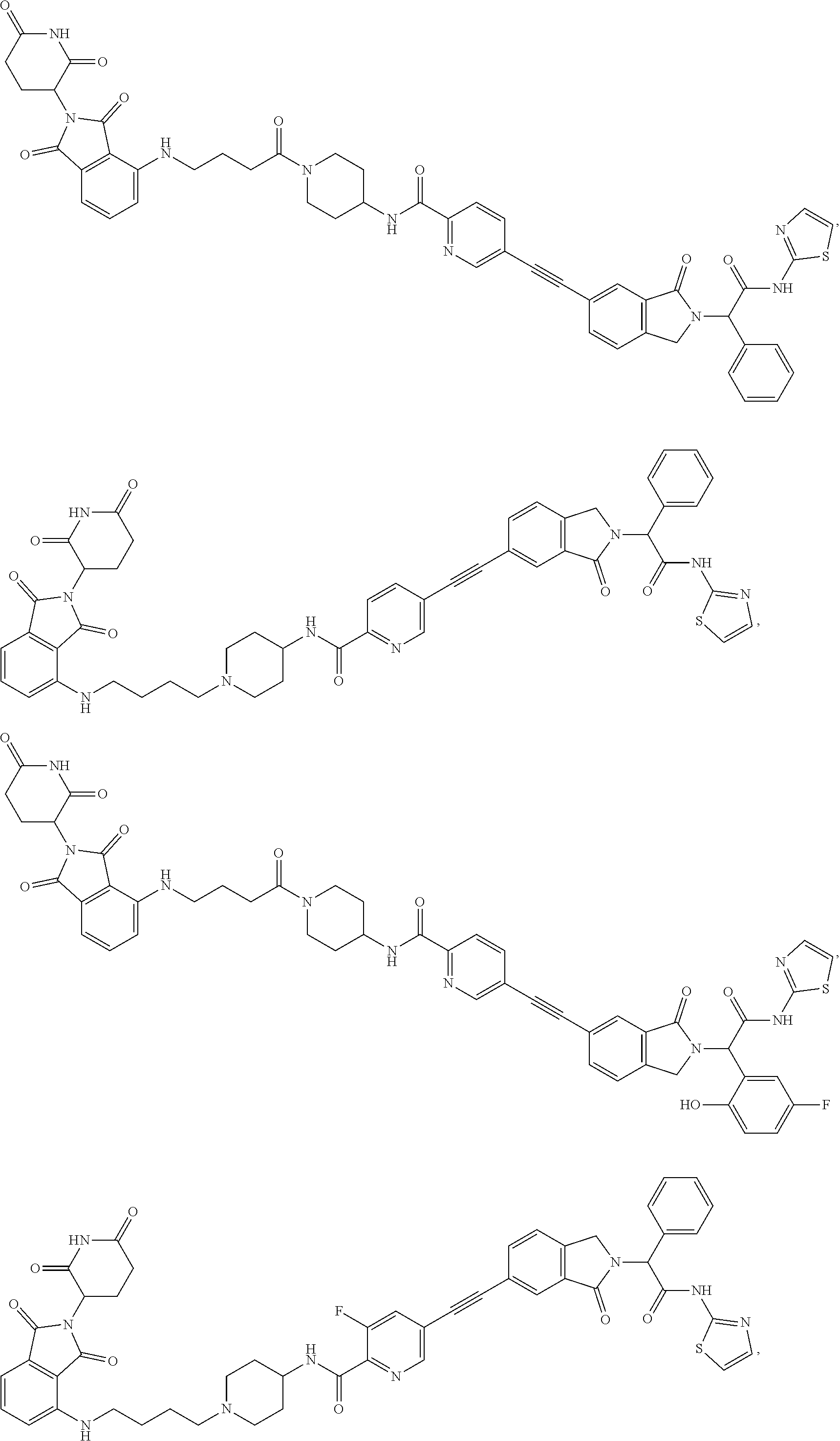 [SMILES: O=C1CCC(N2C(=O)c3cccc(NCCCC(=O)N4CCC(NC(=O)c5ccc(C#Cc6ccc7c(c6)C(=O)N(C(C(=O)Nc6nccs6)c6ccccc6)C7)cn5)CC4)c3C2=O)C(=O)N1 ; O=C1CCC(N2C(=O)c3cccc(NCCCCN4CCC(NC(=O)c5ccc(C#Cc6ccc7c(c6)C(=O)N(C(C(=O)Nc6nccs6)c6ccccc6)C7)cn5)CC4)c3C2=O)C(=O)N1 ; O=C1CCC(N2C(=O)c3cccc(NCCCC(=O)N4CCC(NC(=O)c5ccc(C#Cc6ccc7c(c6)C(=O)N(C(C(=O)Nc6nccs6)c6cc(F)ccc6O)C7)cn5)CC4)c3C2=O)C(=O)N1 ; O=C1CCC(N2C(=O)c3cccc(NCCCCN4CCC(NC(=O)c5ncc(C#Cc6ccc7c(c6)C(=O)N(C(C(=O)Nc6nccs6)c6ccccc6)C7)cc5F)CC4)c3C2=O)C(=O)N1]

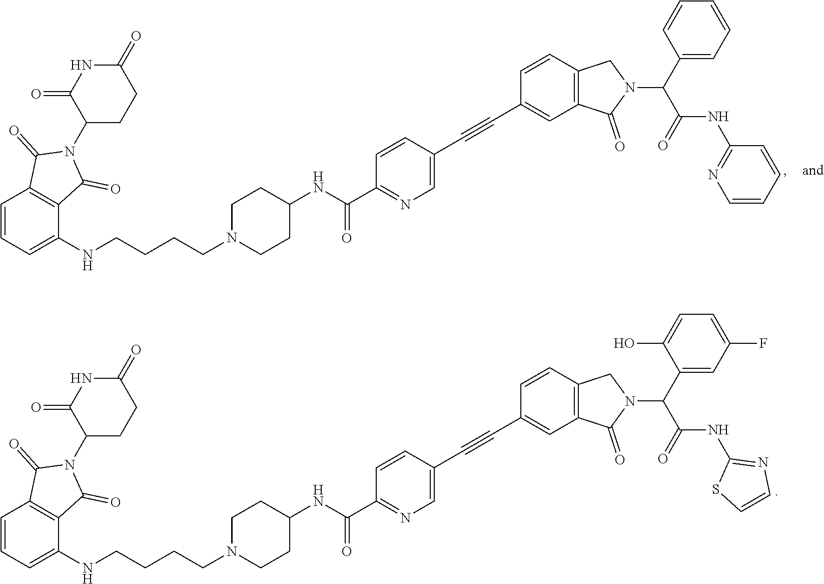 [SMILES: O=C1CCC(N2C(=O)c3cccc(NCCCCN4CCC(NC(=O)c5ccc(C#Cc6ccc7c(c6)C(=O)N(C(C(=O)Nc6ccccn6)c6ccccc6)C7)cn5)CC4)c3C2=O)C(=O)N1 ; O=C1CCC(N2C(=O)c3cccc(NCCCCN4CCC(NC(=O)c5ccc(C#Cc6ccc7c(c6)C(=O)N(C(C(=O)Nc6nccs6)c6cc(F)ccc6O)C7)cn5)CC4)c3C2=O)C(=O)N1]

15. A pharmaceutical composition comprising a compound of claim 1, and a therapeutically inert carrier.

16. A method for the therapeutic or prophylactic treatment of cancer comprising administering an effective amount of a compound of claim 1, or a pharmaceutically acceptable salt thereof, to a patient in need thereof.

17. The method of claim 16, wherein the patient is a human.

18. The method of claim 17, wherein the cancer is EGFR-mediated.
